(19) United States
(12) Patent Application Publication
(10) Pub. No.: US 2022/0193240 A1
(43) Pub. Date: Jun. 23, 2022

(54) TRIGGERABLE HYDROGEL COMPOSITIONS AND RELATED METHODS

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Jinyao Liu, Shanghai (CN); Robert S. Langer, Newton, MA (US)

(73) Assignees: Massachusetts Institute of Technology, Cambridge, MA (US); The Brigham and Women's Hospital, Inc., Boston, MA (US)

(21) Appl. No.: 17/558,867

(22) Filed: Dec. 22, 2021

Related U.S. Application Data
(63) Continuation of application No. 15/758,309, filed on Mar. 7, 2018, now abandoned.
(60) Provisional application No. 62/525,078, filed on Jun. 26, 2017.
(60) Provisional application No. 62/419,650, filed on Nov. 9, 2016.

(51) Int. Cl. A61K 47/36; A61K 9/00; A61K 9/48; A61K 47/32

(52) U.S. Cl. CPC A61K 47/36; A61K 9/0065; A61K 9/06; A61K 47/32; A61K 9/4816

(57) ABSTRACT

Triggerable hydrogel compositions and related methods are generally disclosed. In some embodiments, the compositions and related methods may be used for medical-related or other applications. For example, the compositions and methods described herein may be useful, for example, in biomedical applications such as articles for (e.g., gastric) retention. In some embodiments, methods for deploying and/or removing an article comprising the composition, such as an article for gastric retention, are provided. The article and/or composition may be removed internally from a subject by, for example, introducing at least one reagent (e.g., one reagent, two reagents) such that at least a portion of the composition disassociates. In certain embodiments, the composition comprises a polymer network comprising two or more interpenetrating polymers. In some cases, a first polymer comprises a first cross-link moiety configured to dissociate upon interaction with a reagent. For example, the composition may be administered to a subject such that it is retained at a location internal (e.g., gastric) to the subject. In some embodiments, a reagent may be administered to the subject (e.g., the subject drinks the reagent) such that the reagent interacts with the composition and at least a first cross-link moiety disassociates. In some embodiments, upon disassociation of one or more cross-link moieties of the polymer network, the composition is no longer retained at the location internal to the subject (e.g., dissociates such that it exits the subject). In some cases, the polymer network is configured (e.g., upon administration of the composition to a subject) such that the composition is retained at the location internal to the subject for greater than or equal to 24 hours.

### RELATED APPLICATIONS

This application is a continuation of U.S. application Ser. No. 15/758,309, filed Mar. 7, 2018, and entitled “TRIGGERABLE HYDROGEL COMPOSITIONS AND RELATED METHODS,” which is a national stage application under 35 U.S.C. § 371 of International Patent Application Serial No. PCT/US2017/060932, filed on Nov. 9, 2017, entitled “TRIGGERABLE HYDROGEL COMPOSITIONS AND RELATED METHODS,” which claims priority under 35 U.S.C. § 119(e) to U.S. Provisional Application No. 62/419,650, filed Nov. 9, 2016, and entitled “Triggerable Tough Hydrogels for Gastric Resident Dosage Forms”, and to U.S. Provisional Application No. 62/525,078, filed Jun. 26, 2017, and entitled “Triggerable Hydrogel Compositions And Related Methods,” each of which is incorporated herein by reference in its entirety for all purposes.

### STATEMENT OF GOVERNMENT SUPPORT

This invention was made with Government support under grant number R37 EB000244 awarded by the National Institutes of Health. The Government has certain rights in the invention.

### FIELD OF THE INVENTION

Embodiments described herein generally relate to triggerable hydrogel compositions and related methods.

### BACKGROUND OF THE INVENTION

Drug efficacy is dependent on adherence of a patient to medication. In spite of health risks associated with poor medical adherence, nearly half of patients do not adhere to their prescribed regimen. Delivery devices enabling extended release provide a potential solution to this problem by allowing the administration of a single dose, which would release drugs over a prolonged period of time. However, a key challenge that remains is the on-demand exit from the body and safe passage through the lower gastrointestinal tract when drug administration is no longer required. Accordingly, new materials and methods are needed.

### SUMMARY OF THE INVENTION

Triggerable hydrogel compositions and related methods are generally provided. In one aspect, compositions are provided. In some embodiments, the composition comprises a polymer network comprising first and second interpenetrating polymers and a first cross-link moiety associated with the first polymer, configured to disassociate upon interaction with a first reagent, wherein the composition has a first configuration having an average cross-sectional dimension of less than or equal to 30 cm, and wherein the composition has a second configuration, different than the first configuration such that the composition is retained at a location internal to a subject for greater than or equal to 24 hours in the second configuration. In some embodiments, the composition comprises a second cross-link moiety associated with the second polymer, configured to disassociate upon interaction with a second reagent different than the first reagent.

In another aspect, methods are provided. In some embodiments, the method comprises administering, to a subject, a composition comprising a polymer network comprising first and second interpenetrating polymers, wherein the composition is configured to be retained at a location internal to a subject for greater than or equal to 24 hours and administering, to the subject, a first reagent, such that the first reagent disassociates a first cross-link moiety associated with the first polymer.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows an schematic illustration of the formation and disassociation of an exemplary composition, according to one set of embodiments;

FIG. 1B shows a schematic illustration of an exemplary composition for prolonged drug delivery in the gastric environment, according to one set of embodiments;

FIG. 1C shows a schematic illustration of an exemplary composition comprising alginate and polyacrylamide networks that are interpenetrating, and separately crosslinked by stimuli-responsive Ca2+ ionic and disulfide bonds, e.g., which can be dissolved into solution with a biocompatible chelator and reducing agent, according to one set of embodiments;

FIG. 2A shows photographs of an exemplary composition stretched to 14 times its initial length and subsequently coiled and twisted, and an exemplary composition cuboid resisted slicing with a blade, according to one set of embodiments;

FIG. 2B shows a plot of stress-strain of an exemplary composition, alginate, and polyacrylamide gels with same amounts of alginate or polyacrylamide to the exemplary composition, according to one set of embodiments;

FIG. 2C shows a plot of tensile stress-strain of an exemplary composition, alginate, and polyacrylamide gels stretched to breaking, according to one set of embodiments;

FIG. 2D shows a plot of volume variation (Vt/V0) of an exemplary composition versus incubation time at 37° C., according to one set of embodiments;

FIG. 2E shows a plot of maximum compressive stress of an exemplary composition as a function of the incubation time in simulated gastric fluid (SGF) at 37° C., according to one set of embodiments;

FIG. 2F shows a plot of diameter variation of a cylindrical dehydrated exemplary composition versus incubation time at 37° C., according to one set of embodiments;

FIG. 3A shows a plot of compressive stress of an exemplary composition at strain of 80% versus incubation time with EDTA and GSH at 37° C., according to one set of embodiments;

FIG. 3B shows photographs of an exemplary composition dissolved into viscous solution after 1 h incubation with 80 mM of EDTA and 20 mM of GSH, according to one set of embodiments;

FIG. 3C shows photographs of an exemplary composition before administration, and the retrieved composition after 1 h residence in the gastric cavity of the control and triggered pigs, respectively, according to one set of embodiments;

FIG. 3D shows endoscopy images of an exemplary composition in the stomach from the control and triggered pigs, respectively, according to one set of embodiments. The pigs were treated with 40 mM of EDTA and 20 mM of GSH after delivery of the exemplary composition through the oesophagus. Control animals did not receive EDTA/GSH;

FIG. 4A shows x-ray images of an exemplary composition residing in the gastric cavity of a Yorkshire pig, according to one set of embodiments;

FIG. 4B is a plot of remaining percentage of the intact composition of FIG. 4A in the pig stomach monitored by X-ray imaging versus time post-administration (the inset represents endoscopic image of the composition after 8 days retention in the gastric cavity), according to one set of embodiments;

FIG. 4C is a plot of blood drug concentration as a function of time post-administration for free lumefantrine, according to one set of embodiments;

FIG. 4D is a plot of blood drug concentration as a function of time post-administration for lumefantrine delivered in a lumefantrine-loaded is a plot of blood drug concentration as a function of time post-administration, according to one set of embodiments. In the pig experiments, one composition per pig was implanted at day 0 through the oesophagus;

FIG. 5A shows an HPLC plot of aqueous solutions extracted from an exemplary composition before purification, according to one set of embodiments;

FIG. 5B shows an HPLC plot of aqueous solutions extracted from an exemplary composition after purification to show the complete removal of the unreacted acrylamide from the composition, according to one set of embodiments;

FIG. 6 shows a plot of a cyclic tensile test for an exemplary composition, according to one set of embodiments. Samples of the composition were subjected to a cycle of loading and unloading of varying maximum stretch, according to one set of embodiments;

FIG. 7A shows a plot of tensile stress-strain curves of exemplary compositions incubated in SGF at 37° C. for 4, 8, and 12 days, according to one set of embodiments;

FIG. 7B shows photographs of an exemplary cylindrical composition sample dehydrated in air to 10 times its initial volume, according to one set of embodiments;

FIG. 8A is a representative SEM image of an exemplary composition dehydrated in air, according to one set of embodiments;

FIG. 8B is a representative SEM image of an exemplary composition dehydrated by lyophilization, according to one set of embodiments;

FIG. 8C shows images of the exemplary composition of FIG. 8B before and after dehydration by lyophilization, according to one set of embodiments;

FIG. 9 shows a plot of a compressive stress-strain curve of an exemplary composition after a cycle of complete dehydration and subsequent rehydration, according to one set of embodiments;

FIG. 10A shows a plot of diameter variation of an exemplary composition encapsulated with CaCO₃ inside (thickness of TTH: 1 mm) versus incubation time at 37° C. in SGF, according to one set of embodiments;

FIG. 10B shows images of an exemplary composition encapsulated with CaCO₃ inside expanded in SGF at 37° C., according to one set of embodiments;

FIG. 10C shows images of an exemplary cylindrical composition with 5 wt % CaCO₃ loading floated within 15 min in SGF at 37° C., according to one set of embodiments;

FIG. 11 shows a plot of cell viability for cells cultured in medium incubated with an exemplary composition at 37° C. for 24 h with a dosage range from 0.2 to 50 mg mL−1, according to one set of embodiments. The cells were incubated in the medium for 24 h;

FIG. 12A shows images of a co-culture of an exemplary composition and mouse Lgr5+ intestinal stem cells showed low cytotoxicity of the composition with stem cells over the course of 5 days, according to one set of embodiments;

FIG. 12B shows images of incubation of Lgr5+ stem cells on and within the exemplary composition indicating the cells retained their ability of multilineage differentiation to form organoids, according to one set of embodiments;

FIG. 13 shows a plot of compressive stress of an exemplary composition at strain of 80% versus the incubation time with 20 mM of EDTA or GSH at 37° C., according to one set of embodiments;

FIG. 14 shows a plot of compressive stress of an exemplary composition at strain of 80% versus the incubation time with EDTA and GSH in a range of concentration from 20 to 80 mM at 37° C., according to one set of embodiments;

FIG. 15 shows a plot of GPC measurement of a dissolved exemplary composition, according to one set of embodiments. GPC curves of the dissociated polymers from the composition triggered by EDTA and GSH;

FIG. 16 shows a plot of cell viability for cells cultured for 24 h in the medium with dissociated composition over a concentration range from 0.02 to 5 mg mL−1, according to one set of embodiments;

FIG. 17 shows a plot of compressive stress-strain of an exemplary composition retrieved from a control pig, according to one set of embodiments;

FIG. 18 shows a plot of diameter for a dehydrated exemplary composition loaded with barium sulfide undergoing rehydration in SGF at 37° C., according to one set of embodiments;

FIG. 19A shows representative x-ray images of an exemplary composition disassociating in the gastric cavity of a Yorkshire pig, according to one set of embodiments;

FIG. 19B shows resulting fragments in the intestines of the Yorkshire pig of FIG. 19A as well as the safe pass of the fragments through the intestines in 24 h, according to one set of embodiments;

FIG. 20A shows a plot of tensile stress-strain of an exemplary composition loaded with various wt % of lumefantrine, according to one set of embodiments;

FIG. 20B shows a plot of compressive stress-strain of an exemplary composition loaded with various wt % of lumefantrine, according to one set of embodiments;

FIG. 21A shows a plot of kinetics of release from a lumefantrine-loaded composition in SGF at 37° C., according to one set of embodiments;

FIG. 21B shows a plot of swelling kinetics of a drug-loaded composition in SGF at 37° C., according to one set of embodiments;

FIG. 21C shows an exemplary preparation scheme for a hydrophilic rifampicin-loaded composition, according to one set of embodiments;

FIG. 22A shows a plot of penetration amount through an exemplary composition membrane (thickness: 3 mm) versus incubation time, according to one set of embodiments;

FIG. 22B shows a plot of calculated permeability of DMSO, rifampicin, and insulin, respectively, according to one set of embodiments;

FIG. 23A shows a pharmacokinetic model used to fit to pharmacokinetic data of free lumefantrine, according to one set of embodiments; and

FIG. 23B shows a pharmacokinetic model used to fit to pharmacokinetic data of a lumefantrine-loading composition, according to one set of embodiments.

### DETAILED DESCRIPTION

Triggerable hydrogel compositions and related methods are generally disclosed. In some embodiments, the compositions and related methods may be used for medical-related or other applications. For example, the compositions and methods described herein may be useful, for example, in biomedical applications such as articles for (e.g., gastric) retention. In some embodiments, methods for deploying and/or removing an article comprising the composition, such as an article for gastric retention, are provided. The article and/or composition may be removed internally from a subject by, for example, introducing at least one reagent (e.g., one reagent, two reagents) such that at least a portion of the composition disassociates. In certain embodiments, the composition comprises a polymer network comprising two or more interpenetrating polymers. In some cases, a first polymer comprises a first cross-link moiety configured to dissociate upon interaction with a reagent. For example, the composition may be administered to a subject such that it is retained at a location internal (e.g., gastric) to the subject. In some embodiments, a reagent may be administered to the subject (e.g., the subject drinks the reagent) such that the reagent interacts with the composition and at least a first cross-link moiety disassociates. In some embodiments, upon disassociation of one or more cross-link moieties of the polymer network, the composition is no longer retained at the location internal to the subject (e.g., dissociates such that it exits the subject). In some cases, the polymer network is configured (e.g., upon administration of the composition to a subject) such that the composition is retained at the location internal to the subject for greater than or equal to 24 hours. The composition may be molded into any suitable shape.

Certain embodiments of compositions described herein may offer certain advantages as compared to traditional compositions configured for internal retention and/or drug release, for example, in their ability to adopt a shape and/or size small enough to be ingested by a subject; adopt a shape and/or size internally that slows or prevents further transit in a body cavity (e.g., a gastric cavity); be loaded at high levels (e.g., high mass fraction) with therapeutic, diagnostic, and/or enhancement agents; facilitate controlled release of such therapeutic, diagnostic, and/or enhancement agents with low to no potential for burst release; maintain activity/stability of such therapeutic, diagnostic, and/or enhancement agents in a hostile environment such as the gastric environment for an extended duration (e.g., greater than or equal to 24 hours); maintain safety with low to no potential for gastric or intestinal obstruction and/or perforation; and/or disassociate on demand (e.g., upon administration of one or more reagents) for passing through a gastrointestinal tract. In certain embodiments, the compositions described herein can be configured with durable residence times greater than at least twenty-four hours and lasting up to about one year, or more. In some embodiments, the compositions described herein are compatible (e.g., biocompatible) with subjects, including, but not limited to, humans and non-human animals. In further embodiments, the compositions can be configured to deliver a wide variety of therapeutic, diagnostic, and/or enhancement agents, thus potentially increasing and even maximizing patient treatment therapy adherence rates.

The compositions, articles, and methods described herein offer several advantages over traditional materials (e.g., dissolvable materials) and traditional articles for retention, including the ability to retain the composition at a location internal to the subject for greater than or equal to 24 hours, remove the composition from a location internal to a subject on demand (e.g., upon ingestion of one or more reagents) and/or induce the exit of the composition internal to the subject. The compositions, reagents, and/or articles described herein are generally biocompatible. The compositions and articles described herein may be loaded with bioactive compounds such as drugs and/or folded into a capsule for oral delivery.

The composition may be retained internally of the subject in locations such as, for example, the stomach, the bladder, the esophagus, the colon, the duodenum, the ileum, the jejunum, or the like. In a particular embodiments, the composition is a gastric retention composition. In some embodiments, the composition is configured (e.g., has at least one configuration) such that an average cross-sectional dimension of the composition is substantially similar (e.g., within 10%) of an average cross-sectional dimension of the location internal to the subject. In an exemplary embodiment, the composition comprises a configuration having an average cross-sectional dimension substantially similar to the average cross-sectional dimension of the subject's colon, such that the composition is retained at the colon of the subject for at least 24 hours (e.g., until removed).

The term “subject,” as used herein, refers to an individual organism, for example, a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human. In some embodiments, the subject is a rodent, a mouse, a rat, a hamster, a rabbit, a dog, a cat, a cow, a goat, a sheep, or a pig.

Certain embodiments comprise administering (e.g., orally) a composition comprising a polymer network to a subject such that the composition is retained at a location internal to the subject for a particular amount of time (e.g., at least about 24 hours) before being released or partially released (e.g., upon ingestion of one or more reagents). The composition may be, in some cases, a gastric residence structure. In some embodiments, the compositions described herein comprise one or more materials configured to load an active substance (e.g., an active pharmaceutical ingredient, in some cases at relatively high levels), provide composition stability in acidic environments, mechanical flexibility and strength when contained in an internal cavity (e.g., gastric cavity), easy passage through the GI tract until delivery to a desired internal cavity (e.g., gastric cavity), and/or rapid dissociation upon administration of one or more (e.g., two or more) reagents. In some embodiments, the compositions described herein (e.g, hydrogels) have sufficient mechanical properties (e.g., maximum compressive stress, tensile strength, fracture strain) such that the composition may be retained (e.g., for at least 24 hours) in a gastric environment (e.g., until triggered to disassociate). By contrast, conventional hydrogels may generally suffer from being relatively weak and therefore can be easily broken by, for example, the significant compressive and shearing forces of physiological environments such as the gastric tract, limiting their stability in such an environment and/or lack the capacity to be triggered to disassociate on demand in physiological environments.

In some embodiments, the composition (e.g., a hydrogel) comprises an interpenetrating polymer network comprising at least a first and second interpenetrating polymers. In certain embodiments, the first polymer comprises at least a first cross-link moiety. For example, the interpenetrating polymer network may be formed by mixing two or more monomers (or oligomers, or polymers, or prepolymers) and one or more crosslinking reagents (e.g., a bifunctional monomer, a polyfunctional monomer) such that a first monomer reacts forming a first polymer comprising a first crosslink moiety (e.g., comprising at least a portion of a first crosslinking reagent) and/or a second monomer reacts forming a second polymer comprising a second crosslink moiety (e.g., comprising at least a portion of a second crosslinking reagent).

As used herein, the term “polymer network” refers to a three dimensional substance having oligomeric or polymeric strands interconnected to one another by crosslinks. One of ordinary skill will appreciate that many oligomeric and polymeric compounds are composed of a plurality of compounds having differing numbers of monomers. Such mixtures are often designated by the number average molecular weight of the oligomeric or polymeric compounds in the mixture.

The phase “interpenetrating polymer network,” as used herein, is given its ordinary meaning in the art and generally refers to a polymer network comprising two or more polymer strands in which at least two polymers are at least partially interlaced with one another, such that the network cannot be separated unless chemical bonds are broken. In some embodiments, the at least two polymers interlaced with one another are not (chemically) bonded (e.g., covalently) to each other. In certain embodiments, a first polymer of the at least two polymers interlaced with one another comprises a first crosslinking moiety (e.g., the first polymer is at least partially crosslinked with itself). In some embodiments, a second polymer of the at least two polymers interlaced with one another comprises a second crosslinking moiety (e.g., the second polymer is at least partially crosslinked with itself).

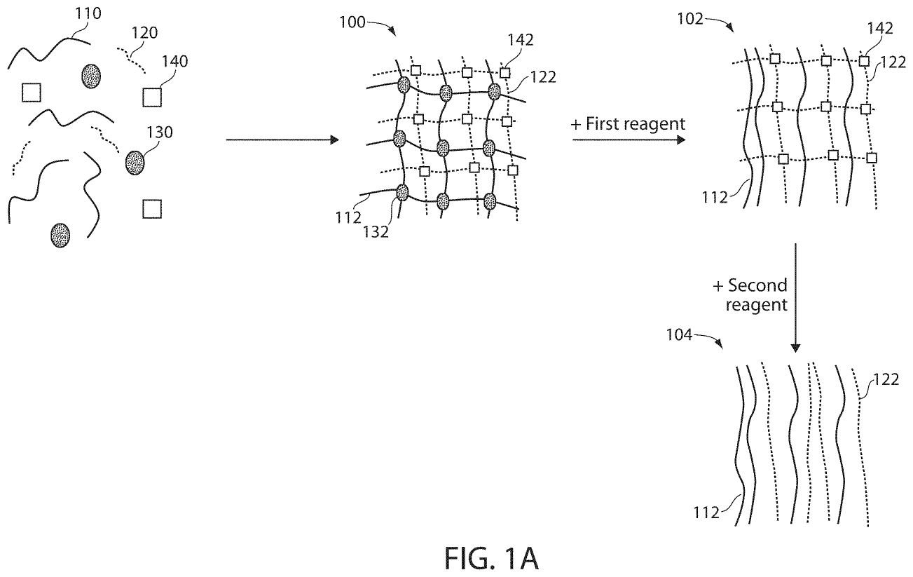

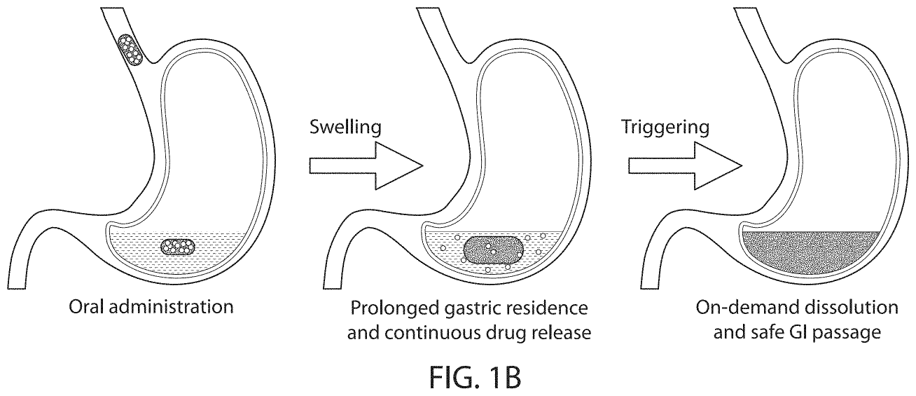

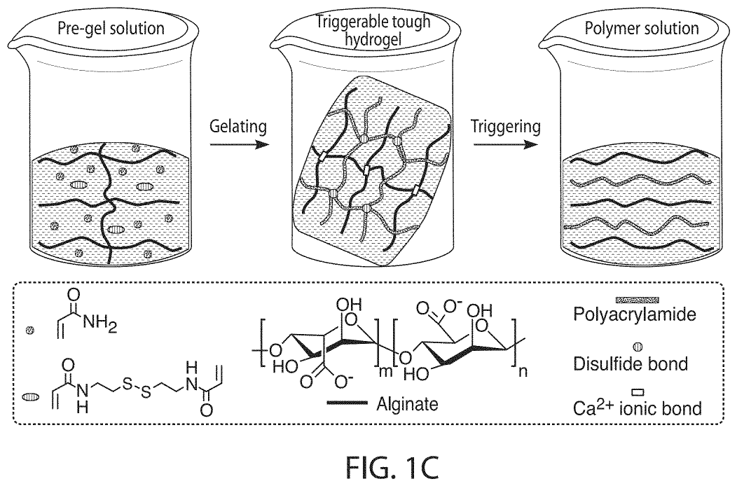

In an exemplary illustrative embodiment, as shown in FIG. 1A, polymer network 100 may be formed by the reaction of monomer (or polymer) 110 with crosslinking reagent 130 and the reaction of monomer (or polymer) 120 with crosslinking reagent 140. In some embodiments, polymer network 100 comprises first polymer 112 (e.g., formed from the reaction of monomer 110 and/or crosslinking reagent 130) and second polymer 122 (e.g., formed from the reaction of monomer 120 and/or crosslinking reagent 140) interpenetrating with first polymer 112. In certain embodiments, first polymer 112 comprises a first crosslinking moiety 132 and/or second polymer 122 comprises a second crosslinking moiety 142.

As used herein, the term “crosslink” refers to a connection between two polymer strands, or a connection between two points one a single polymer strand. The crosslink may either be a chemical bond, a single atom, or multiple atoms. The crosslink may be formed by reaction of a pendant group in one polymer strand with the backbone of a different polymer strand, or by reaction of one pendant group with another pendant group. Crosslinks may exist between separate polymer strands, and may also exist between different points of the same polymer strand. As used herein, the term “polymer strand” refers to an oligomeric or polymeric chain of one monomer unit, or an oligomeric or polymeric chain of two or more different monomer units. As used herein, the term “prepolymer” refers to oligomeric or polymeric strands which have not undergone crosslinking to form a network.

As used herein, the term “crosslink moiety” or “crosslinking moiety” refers to the bond or atom(s) making up the crosslink between two polymer strands (or between different points on the same polymer strand). In some embodiments, the crosslink moiety comprises one or more chemical bonds, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. The ionic bond may comprise, for example, a polyvalent cation. Non-limiting examples of polyvalent cations include calcium, barium, strontium, iron, aluminum. Other polyvalent cations are also possible. In an exemplary embodiment, the polyvalent cation is calcium.

In some embodiments, the crosslink moiety may be formed by mixing a polymer (or polymer precursor and/or monomer) with a crosslinking agent. Non-limiting examples of suitable crosslinking agents include diamine crosslinkers, dicarboxyl crosslinkers, disulfhydryl crosslinkers, dicarbonyl crosslinkers, disulfide crosslinkers, carbodiimide, NHS ester, imidoester, maleimide, haloacetyls, pryidyldisulfide, thiosulfonate, hydrazide, calcium sulphate and N,N′-bis(acryloyl)cystamine. In an exemplary embodiment, the first crosslink moiety is formed from calcium sulphate (e.g., for a crosslink moiety comprising an ionic bond comprising calcium) and the second crosslink moiety is formed from a disulfide crosslinker such as N,N′-bis(acryloyl)cystamine (e.g., for a crosslink moiety comprising a covalent bond such as a disulfide bond). Other crosslinking agents are also possible and those of ordinary skill in the art would be capable of selecting suitable crosslinking agents based upon the teachings of this specification.

As used herein, the term “hydrogel” refers to a polymer network capable of absorbing a relatively high amount of water (e.g., a high weight percentage of water as compared to the weight of the polymer network e.g., greater than 70 wt % water). Referring again to FIG. 1A, in some embodiments, first crosslink moiety 132 may be selected such that, upon interaction of first crosslink moiety 132 with a first reagent, first crosslink moiety 132 disassociates (e.g., illustrated as polymer network 102). In certain embodiments, a second reagent may be added such that second crosslink moiety 142 disassociates (e.g., illustrated as polymer network 104). In some cases, the polymer network may exit the location internal of the subject upon administration of the first reagent and/or the second reagent.

In some cases, the first reagent and the second reagent may be the same (e.g., the first crosslink moiety and the second crosslink moiety are selected such that each dissociates upon exposure to the same reagent). In certain embodiments, the first reagent and the second reagent are different. For example, in some such embodiments, the first reagent at least partially disassociates the first crosslink moiety but does not substantially disassociate the second crosslink moiety. In some embodiments, the second reagent at least partially disassociates the second crosslink moiety.

In an exemplary embodiment, the polymer network comprises first and second interpenetrating polymers, the first polymer comprising a first crosslink moiety and the second polymer comprising a second crosslink moiety, different than the first crosslink moiety. In some embodiments, the first crosslink moiety and the second crosslink moiety each comprises a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. In some embodiments, the first crosslink moiety comprises a first type of bond (e.g., a covalent bond) and the second crosslink moiety comprises a second type of bond (e.g., an ionic bond) different than the first type of bond. In some cases, the first crosslink moiety and the second crosslink moiety are different types of covalent bonds. In an exemplary embodiment, the first crosslink moiety comprises an ionic bond (e.g., comprising a polyvalent cation such as calcium) and the second crosslink moiety comprises a covalent bond (e.g., a disulfide bond). In some embodiments, a crosslink moiety (e.g., the first crosslink moiety, the second crosslink moiety) may be disassociated by breaking the bond (e.g., the covalent bond, the ionic bond), as described herein.

In some embodiments, the polymer network comprises polymers, networks of polymers, and/or multi-block combinations of polymer segments, that may comprise polymers or polymer segments that are for example: polyesters—such as including but not limited to, polycaprolactone, poly(propylene fumarate), poly(glycerol sebacate), poly(lactide), poly(glycol acid), poly(lactic-glycolic acid), polybutyrate, and polyhydroxyalkanoate; polyethers—such as including but not limited to, poly(ethylene oxide) and poly(propylene oxide); polysiloxanes—such as including but not limited to, poly(dimethylsiloxane); polyamides—such as including but not limited to, poly(caprolactam); polyolefins—such as including but not limited to, polyethylene; polycarbonates; polyketals; polyvinyl alcohols; polyoxetanes; polyacrylates/methacrylates—such as including but not limited to, poly[oligo(ethylene glycol) methyl ether methacrylate], poly(2-hydroxyethyl methacrylate) and polyvinylpyrrolidone; polyanhydrides (e.g., polysebacic anhydride); polyacrylamides; polyacrylic acids; polyurethanes; polypeptides; polyphosphoesters; and polysaccharaides—such as including but not limited to, alginate, cellulose, curdlan, dextran, gellan, hyalouran, levan, xanthan pullulan, arabinoxylan, chitin, pectin, and chitosan. In an exemplary embodiment, a first polymer comprises polyacrylamide and a second polymer comprises a polysaccharide such as alginate.

The compositions described herein may be controllably disassociated (e.g., upon introduction of one or more reagents). In some embodiments, each reagent is selected such that it disassociates (e.g., breaks) a particular type of bond. For example, in some embodiments, one or more reagents may be selected to and/or configured to disassociate an ionic bond. Non-limiting examples of reagents suitable for disassociating ionic bonds (e.g., comprising polyvalent cations) include chelating agents (e.g., which may be capable of binding with one or more polycations such as a metal ion). Those of ordinary skill in the art would be capable of selecting other reagents suitable for disassociating ionic bonds based upon the teachings of this specification.

For example, a interpenetrating polymer network comprising a first polymer comprising a first crosslink moiety, and a second polymer comprising a second crosslink moiety, is exposed to a reagent (e.g., the reagent is introduced to the interpenetrating polymer network) such that the first crosslink moiety disassociates.

In some embodiments, the polymer network is present at a location internal to a subject and a reagent (e.g., the chelating agent) is administered (e.g., orally) to the subject such that the reagent interacts with the polymer network and at least partially disassociates at least a first crosslink moiety (e.g., such that the number of crosslinks of the first polymer is reduced). Non-limiting examples of suitable chelating agents include ethylenediaminetetraacetic acid (EDTA), iminodisuccinic acid, polyaspartic acid, ethylenediamine-N,N′-disuccinic acid, Prussian blue, dimercaprol, penicillamine, alpha lipoic acid, BAPTA (1,2-bis(o-aminophenoxy)ethane-N,N,N′,N′-tetraacetic acid), 2,3-Dimercapto-1-propanesulfonic acid, dimercaptosuccinic acid, pentetic acid, egtazic acid, deferasirox, deferiprone, and deferoxamine. Other chelating agents are also possible.

In certain embodiments, one or more reagents may be selected to and/or configured to disassociate a bond (e.g., a covalent bond, an ionic bond). Without wishing to be bound by theory, in some cases, the one or more reagents may disassociate the bond via a chemical reaction that facilitates the disassociation of the bond. For example, in some embodiments, the reagent may comprise a reducing agent. In some cases, a reducing agent may be selected to disassociate a covalent bond such as a disulfide bond. Non-limiting examples of suitable reducing agents include L-glutathione, dithiothreitol, dithioerythritol, mercaptoethanol, L-cysteine, and tris (2-Carboxyethyl) phosphine hydrochloride. Those of ordinary skill in the art would be capable of selecting other reagents suitable for disassociating covalent bonds based upon the teachings of this specification.

Each reagent may be administered at any suitable concentration (e.g., such that there are no significant adverse effects on the subject). In certain embodiments, the concentrate of the reagent is selected such that the reagent is substantially non-toxic to the subject. The term “toxic” refers to a substance showing detrimental, deleterious, harmful, or otherwise negative effects on a subject, tissue, or cell when or after administering the substance to the subject or contacting the tissue or cell with the substance, compared to the subject, tissue, or cell prior to administering the substance to the subject or contacting the tissue or cell with the substance. In certain embodiments, the effect is death or destruction of the subject, tissue, or cell. In certain embodiments, the effect is a detrimental effect on the metabolism of the subject, tissue, or cell. In certain embodiments, a toxic substance is a substance that has a median lethal dose (LD50) of not more than 500 milligrams per kilogram of body weight when administered orally to an albino rat weighing between 200 and 300 grams, inclusive. In certain embodiments, a toxic substance is a substance that has an LD50 of not more than 1,000 milligrams per kilogram of body weight when administered by continuous contact for 24 hours (or less if death occurs within 24 hours) with the bare skin of an albino rabbit weighing between two and three kilograms, inclusive. In certain embodiments, a toxic substance is a substance that has an LC50 in air of not more than 2,000 parts per million by volume of gas or vapor, or not more than 20 milligrams per liter of mist, fume, or dust, when administered by continuous inhalation for one hour (or less if death occurs within one hour) to an albino rat weighing between 200 and 300 grams, inclusive.

The term “non-toxic” refers to a substance that is not toxic. Toxic reagents include, e.g., oxidative stressors, nitrosative stressors, proteasome inhibitors, inhibitors of mitochondrial function, ionophores, inhibitors of vacuolar ATPases, inducers of endoplasmic reticulum (ER) stress, and inhibitors of endoplasmic reticulum associated degradation (ERAD). In some embodiments a toxic reagent selectively causes damage to nervous system tissue. Toxic reagents include compounds that are directly toxic and reagents that are metabolized to or give rise to substances that are directly toxic. It will be understood that the term “toxic compounds” typically refers to reagents that are not ordinarily present in a cell's normal environment at sufficient levels to exert detectable damaging effects. However, in some cases, the toxic reagents may be present in a cell's normal environment but at concentrations significantly less than present in the auxiliary materials described herein. Typically toxic reagents exert damaging effects when present at a relatively low concentration, e.g., at or below 1 mM, e.g., at or below 500 microM, e.g., at or below 100 microM. It will be understood that a toxic reagents typically has a threshold concentration below which it does not exert detectable damaging effects. The particular threshold concentration will vary depending on the agent and, potentially, other factors such as cell type, other agents present in the environment, etc.

In some embodiments, the concentration of the reagent (e.g., chelating agent, reducing agent) may be selected such that it effectively disassociates a bond while e.g., being substantially non-toxic. In certain embodiments, the concentration of the reagent is greater than or equal to 1 mM, greater than or equal to 2 mM, greater than or equal to 5 mM, greater than or equal to 10 mM, greater than or equal to 12 mM, greater than or equal to 15 mM, greater than or equal to 20 mM, greater than or equal to 25 mM, greater than or equal to 30 mM, greater than or equal to 35 mM, greater than or equal to 40 mM, greater than or equal to 45 mM, greater than or equal to 50 mM, greater than or equal to 55 mM, greater than or equal to 60 mM, greater than or equal to 70 mM, greater than or equal to 80 mM, or greater than or equal to 90 mM. In certain embodiments, the concentration of the regant is less than or equal to 100 mM, less than or equal to 90 mM, less than or equal to 80 mM, less than or equal to 70 mM, less than or equal to 60 mM, less than or equal to 55 mM, less than or equal to 50 mM, less than or equal to 45 mM, less than or equal to 40 mM, less than or equal to 35 mM, less than or equal to 30 mM, less than or equal to 25 mM, less than or equal to 20 mM, less than or equal to 15 mM, less than or equal to 12 mM, less than or equal to 10 mM, less than or equal to 5 mM, or less than or equal to 2 mM. Combinations of the above referenced ranges are also possible (e.g., greater than or equal to 1 mM and less than or equal to 100 mM). Other ranges are also possible.

In some embodiments, the composition has a first configuration (e.g., such that the composition may be encapsulated) and a second configuration (e.g., such that the composition expands and/or may be retained at a location internal to a subject).

In some embodiments, the second configuration may be such that the composition is retained at a location internal of a subject (e.g., for greater than or equal to 24 hours), and the first configuration is constructed and arranged such that the structure may be encapsulated (e.g., for oral delivery of the composition within a capsule). In some cases, the second configuration is sufficiently large such that the structure is retained at a location internal of the subject and the first configuration is sufficiently small such that the structure may fit within a particular size capsule suitable for oral delivery to a subject. The phrase “retained at a location internal of a subject” as used herein generally refers to a composition maintaining its relative position within a subject (e.g., a location in the GI tract such as the colon, the duodenum, the ileum, the jejunum, the stomach, or the esophagus) for a given amount of time (e.g., greater than or equal to 24 hours) e.g., until acted upon such that it is released from the location internal of the subject (e.g., by administration of one or more reagents as described herein). Those of ordinary skill in the art would understand that the phrase “retained at a location” shall be understood to not require absolute conformance to an exact atomistic and/or molecular location within a subject but, rather, shall be understood to indicate retention at or near a specific location to the extent possible for a composition subject to physiological environments and as would be understood by one skilled in the art most closely related to such compositions for retention (e.g., gastric retention).

In certain embodiments, a configuration of the composition may be characterized by a largest dimension (e.g., width, length). In some embodiments, the largest dimension of the first configuration may be at least about 10% less, at least about 20% less, at least about 40% less, at least about 60% less, or at least about 80% less than the largest dimension of the second configuration. In certain embodiments, the largest dimension of the second configuration may be at least about 100% greater, at least about 200% greater, at least about 400% greater, at least about 600% greater, or at least about 800% greater than the largest dimension of the first configuration. Any and all closed ranges that have endpoints within any of the above referenced ranges are also possible (e.g., between about 10% and about 80%, between about 10% and about 40%, between about 20% and about 60%, between about 40% and about 80%). Other ranges are also possible.

In some cases, the composition may have a relatively high aspect ratio such that the largest average cross-sectional dimension of the first configuration is within 10% (e.g., within 5%, within 2%, within 1%) of the largest dimension of the second configuration. In some such embodiments, an average cross-sectional dimension (e.g., diameter) of the first configuration may be at least about 10% less, at least about 20% less, at least about 40% less, at least about 60% less, or at least about 80% less than the average cross-sectional dimension of the second configuration. In certain embodiments, the largest cross-sectional dimension of the second configuration may be at least about 100% greater, at least about 200% greater, at least about 400% greater, at least about 600% greater, or at least about 800% greater than the largest cross-sectional dimension of the first configuration.

In some embodiments, the configuration of the composition may be characterized by a convex hull volume of the structure. The term convex hull volume is known in the art and generally refers to a set of surfaces defined by the periphery of a 3-D object such that the surfaces define a particular volume. In some embodiments, the convex hull volume of the first configuration may be at least about 10% less, at least about 20% less, at least about 40% less, at least about 60% less, or at least about 80% less than the convex hull volume of the second configuration. In certain embodiments, the convex hull volume of the second configuration may be at least about 10% less, at least about 20% less, at least about 40% less, at least about 60% less, or at least about 80% less than the convex hull volume of the first configuration. Any and all closed ranges that have endpoints within any of the above referenced ranges are also possible (e.g., between about 10% and about 80%, between about 10% and about 40%, between about 20% and about 60%, between about 40% and about 80%). Other ranges are also possible.

In certain embodiments, the second configuration is obtained upon swelling of the composition under physiological conditions. For example, the composition may be administered to a subject (e.g., orally) in the first configuration and, upon reaching a desired location internal to a subject (e.g., a gastric cavity), the composition absorbs fluid (e.g., gastric fluid, water) such that it obtains the second configuration (e.g., swells). In some embodiments, the composition in the second configuration comprises greater than or equal to 70 wt % fluid, greater than or equal to 75 wt % fluid, greater than or equal to 80 wt % fluid, greater than or equal to 85 wt % fluid, greater than or equal to 90 wt % fluid, greater than or equal to 95 wt % fluid, or greater than or equal to 98 wt % fluid versus the total weight of the composition. In certain embodiments, the composition in the second configuration comprises less than or equal to 99 wt % fluid, less than or equal to 98 wt % fluid, less than or equal to 95 wt % fluid, less than or equal to 90 wt % fluid, less than or equal to 85 wt % fluid, less than or equal to 80 wt % fluid, or less than or equal to 75 wt % fluid versus the total weight of the composition. Combinations of the above-referenced ranges are also possible (e.g., greater than or equal to 70 wt % and less than or equal to 99 wt %). Other ranges are also possible.

In some cases, the second configuration has a volume that is at least twice the volume of the first configuration. For example, a fluid (e.g., water, phosphate buffer saline, simulated gastric fluid) may be added to the composition in the first configuration and the composition obtains (e.g., swells) the second configuration such that the volume of the second configuration is at least 2, at least 3, at least 4, at least 5, or at least 8 times the volume of the first configuration. In certain embodiments, the volume of the second configuration is less than or equal to 10, less than or equal to 8, less than or equal to 5, less than or equal to 4, or less than or equal to 3 times the volume of the first configuration. Combinations of the above referenced ranges are also possible (e.g., at least 2 and less than or equal to 10). Other ranges are also possible.

In some cases, the first configuration may have a largest dimension, aspect ratio, convex hull volume, and/or volume that is different than a largest dimension, aspect ratio, convex hull volume, and/or volume of the second configuration, respectively.

In some embodiments, the composition in the second configuration has desirable mechanical properties (e.g., for retention at a location internal to the subject for greater than or equal to 24 hours). In some embodiments, the mechanical properties of the structure are optimized for safe transient retention of all or a portion of the structure in an internal cavity such as the gastric cavity for durations greater than 24 hours, including up to about one year or longer. Advantageously, the compositions (e.g., hydrogels) described herein may have mechanical properties suitable for gastric residence as compared to traditional hydrogels which, as described above, may not withstand the compressive and/or shearing forces of physiological environments such as the gastric tract such that they, in some cases, cannot reside at a location internal to a subject for at least 24 hours and/or lack the capacity to be triggered to disassociate on demand in physiological environments.

In certain embodiments, the composition (e.g., before disassociation) has a maximum compressive stress of greater than or equal to 1 MPa, greater than or equal to 1.5 MPa, greater than or equal to 2 MPa, greater than or equal to 2.25 MPa, greater than or equal to 2.5 MPa, greater than or equal to 2.75 MPa, greater than or equal to 3 MPa, greater than or equal to 3.25 MPa, greater than or equal to 3.5 MPa, greater than or equal to 3.75 MPa, greater than or equal to 4 MPa, greater than or equal to 4.5 MPa, greater than or equal to 5 MPa, greater than or equal to 6 MPa, greater than or equal to 7 MPa, greater than or equal to 8 MPa, or greater than or equal to 9 MPa. In some embodiments, the composition has a maximum compressive stress of less than or equal to 10 MPa, less than or equal to 9 MPa, less than or equal to 8 MPa, less than or equal to 7 MPa, less than or equal to 6 MPa, less than or equal to 5 MPa, less than or equal to 4.5 MPa, less than or equal to 4 MPa, less than or equal to 3.75 MPa, less than or equal to 3.5 MPa, less than or equal to 3.25 MPa, less than or equal to 3 MPa, less than or equal to 2.75 MPa, less than or equal to 2.5 MPa, less than or equal to 2.25 MPa, less than or equal to 2 MPa, or less than or equal to 1.5 MPa. Combinations of the above referenced ranges are also possible (e.g., greater than or equal to 1 MPa and less than or equal to 10 MPa, greater than or equal to 2.25 MPa and less than or equal to 4 MPa). Other ranges are also possible.

In some embodiments, the composition has a tensile strength of greater than or equal to 40 kPa, greater than or equal to 50 kPa, greater than or equal to 60 kPa, greater than or equal to 70 kPa, greater than or equal to 80 kPa, greater than or equal to 90 kPa, greater than or equal to100 kPa, greater than or equal to 110 kPa, greater than or equal to 120 kPa, greater than or equal to 130 kPa, greater than or equal to 140 kPa, greater than or equal to 150 kPa, greater than or equal to 160 kPa, greater than or equal to 170 kPa, greater than or equal to 180 kPa, or greater than or equal to 190 kPa. In certain embodiments, the composition has a tensile strength of less than or equal to 200 kPa, less than or equal to 190 kPa, less than or equal to 180 kPa, less than or equal to 170 kPa, less than or equal to 160 kPa, less than or equal to 150 kPa, less than or equal to 140 kPa, less than or equal to 130 kPa, less than or equal to 120 kPa, less than or equal to 110 kPa, less than or equal to 100 kPa, less than or equal to 90 kPa, less than or equal to 80 kPa, less than or equal to 70 kPa, or less than or equal to 60 kPa. Combinations of the above referenced ranges are also possible (e.g., greater than or equal to 40 kPa and less than or equal to 200 kPa, greater than or equal to 50 kPa and less than or equal to 150 kPa). Other ranges are also possible.

In certain embodiments, the composition has a fracture strain of greater than or equal to 5%, greater than or equal to 6%, greater than or equal to 7%, greater than or equal to 8%, greater than or equal to 9%, greater than or equal to 10%, greater than or equal to 11%, greater than or equal to 12%, greater than or equal to 13%, greater than or equal to 14%, greater than or equal to 15%, greater than or equal to 16%, greater than or equal to 17%, greater than or equal to 18%, or greater than or equal to 19%. In some embodiments, the composition has a fracture strain of less than or equal to 20%, less than or equal to 19%, less than or equal to 18%, less than or equal to 17%, less than or equal to 16%, less than or equal to 15%, less than or equal to 14%, less than or equal to 13%, less than or equal to 12%, less than or equal to 11%, less than or equal to 10%, less than or equal to 9%, less than or equal to 8%, less than or equal to 7%, or less than or equal to 6%. Combinations of the above-referenced ranges are also possible (e.g., greater than or equal to 5% and less than or equal to 20%). Other ranges are also possible.

Those skilled in the art given the guidance and teaching of this specification would be capable of determining suitable methods for tuning the mechanical properties of the composition by, for example, varying the molar ratios of monomeric and/or polymeric units, varying cross-linking density, varying the concentration of cross-linking agents used in the formation of the polymer, varying the crystallinity of the polymer (e.g., by varying the ratio of crystalline and amorphous regions in the polymer) and/or the use of additional or alternative materials.

In some embodiments, the composition (e.g., in the first configuration) may be stable under ambient conditions (e.g., room temperature, atmospheric pressure and relative humidity) and/or physiological conditions (e.g., in the second configuration at or about 37° C., in physiologic fluids) for at least about 1 day, at least about 3 days, at least about 7 days, at least about 2 weeks, at least about 1 month, at least about 2 months, at least about 6 months, at least about 1 year, or at least about 2 years. In certain embodiments, the composition may be stable for less than or equal to about 3 years, less than or equal to about 2 years, less than or equal to about 1 year, less than or equal to about 1 month, less than or equal to about 1 week, or less than or equal to about 3 days. Any and all closed ranges that have endpoints within any of the above-referenced ranged are also possible (e.g., between about 24 hours and about 3 years, between about 1 week and 1 year, between about 1 year and 3 years). Other ranges are also possible.

In some embodiments, the composition is loaded (e.g., during and/or after formation of the polymer network of the composition) with an active substance such as a therapeutic, diagnostic, and/or enhancement agents. In other embodiments, the composition is loaded with therapeutic, diagnostic, and/or enhancement agents after it is already retained at a location internal to a subject, such as a gastric cavity. In some embodiments, a composition is configured to maintain stability of therapeutic, diagnostic, and/or enhancement agents in a hostile physiological environment (e.g., the gastric environment) for an extended duration. In further embodiments, the composition is configured to control release of therapeutic, diagnostic, and/or enhancement agents e.g., with low to no potential for burst release. In some embodiments, the composition is pre-loaded and/or loaded with a combination of active substances. For example, in certain embodiments, the structure comprises one or more, two or more, three or more, or four or more active substances.

Therapeutic, diagnostic, and/or enhancement agents can be loaded into the composition via standard methods including, but not limited to, powder mixing, direct addition, solvent loading, melt loading, physical blending, supercritical carbon dioxide assisted, and conjugation reactions such as ester linkages and amide linkages. Release of therapeutic, diagnostic, and/or enhancement agents can then be accomplished through methods including, but not limited to, dissolution of the composition comprising a polymeric matrix material, degradation of the matrix material, swelling of the matrix material, diffusion of an agent, hydrolysis, and chemical or enzymatic cleavage of conjugating bonds. In some embodiments, the active substance is covalently bound to one or more polymers of the polymer network (e.g., and is released while the composition resides at a location internal to a subject).

In certain embodiments, the composition is constructed and arranged to release the active substance from the polymer network. Such embodiments may be useful in the context of drug delivery. In other embodiments, the active substance is permanently affixed to the composition. Such embodiments may be useful in molecular recognition and purification contexts. In certain embodiments, the active substance is embedded within the composition. In some embodiments, the active substance is associated with the composition (e.g., associated with one or more polymers of the polymer network) via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups.

According to some embodiments, the composition and methods described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. The active substance may be entrapped within the polymer network or may be directly attached to one or more polymers in the polymer network through a chemical bond. In certain embodiments, the active substance is covalently bonded to one or more polymers of the polymer network. For example, in some embodiments, the active substance is bonded to a polymer through a carboxylic acid derivative. In some cases, the carboxylic acid derivative may form an ester bond with the active substance.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In some embodiments, the active substance is a radiopaque material such as tungsten carbide or barium sulfate.

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the therapeutic agent is one or more antimalarial drugs. Exemplary antimalarial drugs include quinine, lumefantrine, chloroquine, amodiaquine, pyrimethamine, proguanil, chlorproguanil-dapsone, sulfonamides such as sulfadoxine and sulfamethoxypyridazine, mefloquine, atovaquone, primaquine, halofantrine, doxycycline, clindamycin, artemisinin and artemisinin derivatives. In some embodiments, the antimalarial drug is artemisinin or a derivative thereof. Exemplary artemisinin derivatives include artemether, dihydroartemisinin, arteether and artesunate. In certain embodiments, the artemisinin derivative is artesunate.

Active substances that contain a carboxylic acid group may be directly incorporated into a polymer network that contain ester and hydroxyl groups without further modification. Active substances containing an alcohol may first be derivatized as a succinic or fumaric acid monoester and then incorporated into the p. Active substances that contain a thiol may be incorporated into an olefin or acetylene-containing polymer(s) through a sulfur-ene reaction. In other embodiments, the one or more agents are non-covalently associated with the polymer network (e.g., dispersed or encapsulated within the polymer network). In some such embodiments, the active substance may be dispersed or encapsulated within by hydrophilic and/or hydrophobic forces.

In other embodiments, the active substance is a protein or other biological macromolecule. Such substances may be covalently bound to one or more polymers of the polymer network through ester bonds using available carboxylate containing amino acids, or may be incorporated into polymeric material containing olefinic or acetylenic moieties using a thiol-ene type reaction. In some cases, the active substance comprises an amine functional group capable of reacting with an epoxide functional group to form an amide or ester bond.

The active substance may be associated with the polymer network and/or present in the composition in any suitable amount. In some embodiments, the active substance is present in the composition in an amount ranging between about 0.01 wt % and about 50 wt % versus the total composition weight. In some embodiments, the active substance is present in the composition in an amount of at least about 0.01 wt %, at least about 0.05 wt %, at least about 0.1 wt %, at least about 0.5 wt %, at least about 1 wt %, at least about 2 wt %, at least about 3 wt %, at least about 5 wt %, at least about 10 wt %, at least about 20 wt %, at least about 30 wt %, at least about 40 wt % of the total composition weight. In certain embodiments, the active substance is present in the composition in an amount of less than or equal to about 50 wt %, less than or equal to about 40 wt %, less than or equal to about 30 wt %, less than or equal to about 20 wt %, less than or equal to about 10 wt %, less than or equal to about 5 wt %, less than or equal to about 3 wt %, less than or equal to about 2 wt %, less than or equal to about 1 wt %, less than or equal to about 0.5 wt %, less than or equal to about 0.1 wt %, or less than or equal to about 0.05 wt % versus the total composition weight. Any and all closed ranges that have endpoints within any of the above-referenced ranges are also possible (e.g., between about 0.01 wt % and about 50 wt %). Other ranges are also possible.

Advantageously, certain embodiments of the compositions described herein may permit higher concentrations (weight percent) of active substances such as therapeutic agents to be incorporated as compared to other polymers such as certain conventional hydrogels. In some embodiments, the active substance (e.g., the active substance) may be released from the composition. In certain embodiments, the active substance is released by diffusion out of the composition. In some embodiments, the active substance is released by degradation of the composition (e.g., biodegradation, enzymatic degradation, hydrolysis). In some embodiments, the active substance is released from the composition at a particular rate. Those skilled in the art would understand that the rate of release may be dependent, in some embodiments, on the solubility of the active substance in the medium in which the composition is exposed, such as a physiological fluid such as gastric fluid. The ranges and description included related to the release and/or rate of release of the active substance is generally in reference to hydrophilic, hydrophobic, and/or lipophilic active substances in simulated gastric fluid (e.g., as defined in the United States Pharmacopeia (USP)). Simulated gastric fluids are known in the art and those skilled in the art would be capable of selecting suitable simulated gastric fluids based on the teachings of this specification.

In some embodiments, between 0.05 wt % to 99 wt % of the active substance initially contained in a composition is released (e.g., in vivo) between 24 hours and 1 year. In some embodiments, between about 0.05 wt % and about 99.0 wt % of the active substance is released (e.g., in vivo) from the composition after a certain amount of time. In some embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the active substance associated with the composition is released from the composition (e.g., in vivo) within about 24 hours, within 36 hours, within 72 hours, within 96 hours, or within 192 hours. In certain embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the active substance associated with the composition is released from the composition (e.g., in vivo) within 1 day, within 5 days, within 30 days, within 60 days, within 120 days, or within 365 days. For example, in some cases, at least about 90 wt % of the active substance associated with the composition is released from the composition (e.g., in vivo) within 120 days.

In some embodiments, the active substance is released from the loadable polymeric material at a particular initial average rate as determined over the first 24 hours of release (the “initial rate”) (e.g., release of the active substance at the desired location internally of the subject, such as an internal cavity). In certain embodiments, the active substance is released at an average rate of at least about 1%, at least about 2%, at least about 5%, least about 10%, at least about 20%, at least about 30%, least about 50%, at least about 75%, at least about 80%, at least about 90%, at least about 95%, or at least about 98% of the initial average rate over a 24 hour period after the first 24 hours of release. In some embodiments, the active substance is released at an average rate of less than or equal to about 99%, less than or equal to about 98%, less than or equal to about 95%, less than or equal to about 90%, less than or equal to about 80%, less than or equal to about 75%, less than or equal to about 50%, less than or equal to about%, less than or equal to about 30%, less than or equal to about 20%, less than or equal to about 10%, less than or equal to about 5%, or less than or equal to about 2% of the initial average rate over a 24 hour period after the first 24 hours of release. Any and all closed ranges that have endpoints within any of the above referenced ranges are also possible (e.g., between about 1% and about 99%, between about 1% and about 98%, between about 2% and about 95%, between about 10% and about 30%, between about 20% and about 50%, between about 30% and about 80%, between about 50% and about 99%). Other ranges are also possible.

The active substance may be released at an average rate over at least one selected continuous 24 hour period at a rate of between about 1% and about 99% of the initial rate between 48 hours and about 1 year (e.g., between 48 hours and 1 week, between 3 days and 1 month, between 1 week and 1 month, between 1 month and 6 months, between 3 months and 1 year, between 6 months and 2 years) after the initial release.

For example, in some cases, the active substance may be released at a rate of between about 1% and about 99% of the initial rate on the second day of release, the third day of release, the fourth day of release, the fifth day of release, the sixth day of release, and/or the seventh day of release. In certain embodiments, burst release of an active substance from the composition is generally avoided. In an illustrative embodiment, in which at least about 0.05 wt % of the active substance is released from the composition within 24 hours, between about 0.05 wt % and about 99 wt % is released during the first day of release (e.g., at the location internally of the subject), and between about 0.05 wt % and about 99 wt % is released during the second day of release. Those skilled in the art would understand that the active substance may be further released in similar amounts during a third day, a fourth day, a fifth day, etc. depending on the properties of the composition and/or the active substance.

In certain embodiments, the active substance may be released with a pulse release profile. For example, in some embodiments, the active substance may be released on the first day after administration and during another 24 hour period such as starting during the third day, the fourth day, or the fifth day, but is not substantially released on other days. Those skilled in the art would understand that other days and/or combinations of pulsing and continuous release are also possible.

The active substance may be released at a relatively constant average rate (e.g., a substantially zero-order average release rate) over a time period of at least about 24 hours. In certain embodiments, the active substance is released at a first-order release rate (e.g., the rate of release of the active substance is generally proportional to the concentration of the active substance) of a time period of at least about 24 hours.

In some embodiments, at least a portion of the active substance loaded into the composition is released continuously (e.g., at varying rates) over the residence time period of the composition. Residence time periods are described in more detail herein.

In some embodiments, the composition (e.g., comprising a polymer network) comprises one or more configurations (e.g., a first configuration, a second configuration) as described above. For example, in certain embodiments, the composition has a particular configuration such as a defined shape, size, orientation, and/or volume. The composition may comprise any suitable configuration. In some embodiments, the composition has a particular shape as defined by a cross-sectional area of the composition. Non-limiting examples of suitable cross-sectional shapes include square, circles, ovals, polygons (e.g., pentagons, hexagons, heptagons, octagons, nonagons, dodecagons, or the like), tubes, rings, star or star-like/stellate, or the like. Those skilled in the art would be capable of selecting suitable shapes depending on the application and based upon the teachings of this specification.

In some embodiments, the composition in the first configuration is contained within a capsule and delivered orally to a subject. In some such embodiments, the composition may travel to the stomach and the capsule may release the composition from the capsule, upon which the composition obtains (e.g., swells) the second configuration.

In some embodiments, the average cross-sectional dimension of the second configuration is at least about 0.5 cm, at least about 1 cm, at least about 2 cm, at least about 4 cm, at least about 5 cm, at least about 10 cm, at least about 15 cm, or at least about 20 cm. In certain embodiments, the average cross-sectional dimension of the second configuration is less than or equal to about 30 cm, less than or equal to about 20 cm, less than or equal to about 15 cm, less than or equal to about 10 cm, less than or equal to about 5 cm, less than or equal to about 4 cm, less than or equal to about 2 cm, or less than or equal to about 1 cm. Any and all closed ranges that have endpoints within any of the above-referenced ranges are also possible (e.g., between about 0.5 cm and about 30 cm). Those skilled in the art would be capable of selecting suitable cross-sectional dimensions for compositions based upon the teachings of this specification e.g., for specific orifices of a subject such that the composition is retained (e.g., at a location internal to a subject).

As described herein, in some embodiments, the composition is configured to adopt a shape and/or size compatible with oral administration to and/or ingestion by a subject. In some embodiments, the composition has a shape with a capacity for folding and/or packing into stable encapsulated forms. For example, in some embodiments the composition (e.g., in the first configuration) is designed to maximally pack and fill a capsule or other soluble container (e.g., a containing structure). In some embodiments, the composition has a shape that maximally fills and/or packs into a capsule or other soluble container.

In some embodiments, an article comprises the composition and a containing structure. In certain embodiments, the composition comprises more than 60 vol % of the containing structure. Based on the application, a capsule may be manufactured to particular specifications or a standard size, including, but not limited to, a 000, 00, 0, 1, 2, 3, 4, and 5, as well as larger veterinary capsules Su07, 7, 10, 12e1, 11, 12, 13, 110 ml, 90 ml, and 36 ml. In some embodiments, the structure may be provided in capsules, coated or not. The capsule material may be either hard or soft, and as will be appreciated by those skilled in the art, typically comprises a tasteless, easily administered and water soluble compound such as gelatin, starch or a cellulosic material.

In some embodiments, the article and/or composition is administered to a subject (e.g., orally). In certain embodiments, the article and/or composition may be administered orally, rectally, vaginally, nasally, or uretherally. In an exemplary embodiment, the tissue-interfacing component is administered orally and, upon reaching a location internal the subject (e.g., the GI tract such as the colon, the duodenum, the ileum, the jejunum, the stomach, or the esophagus), the composition is released from encapsulation and/or swells at the location internal the such that the composition is retained at the location (e.g., for greater than or equal to 24 hours). In certain embodiments, at least a portion the active pharmaceutical agent dissolves into the tissue of the subject (e.g., at or proximate the location internal to the subject).

Any terms as used herein related to shape, orientation, alignment, and/or geometric relationship of or between, for example, one or more articles, compositions, structures, materials and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “ square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described.

As used herein, the term “react” or “reacting” refers to the formation of a bond between two or more components to produce a stable, isolable compound. For example, a first component and a second component may react to form one reaction product comprising the first component and the second component joined by a covalent bond. The term “reacting” may also include the use of solvents, catalysts, bases, ligands, or other materials which may serve to promote the occurrence of the reaction between component(s). A “stable, isolable compound” refers to isolated reaction products and does not refer to unstable intermediates or transition states.

The terms “amine” and “amino” refer to both unsubstituted and substituted amines, e.g., a moiety that can be represented by the general formula: N(R′)(R″)(R′″) wherein R′, R″, and R′″ each independently represent a group permitted by the rules of valence.

The terms “acyl,” “carboxyl group,” or “carbonyl group” are recognized in the art and can include such moieties as can be represented by the general formula:

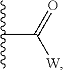

wherein W is H, OH, O-alkyl, O-alkenyl, or a salt thereof. Where W is O-alkyl, the formula represents an “ester.” Where W is OH, the formula represents a “carboxylic acid.” In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

As used herein, the term “thiol” means —SH; the term “hydroxyl” means —OH; and the term “sulfonyl” means —SO₂—.

The term “substituted” is contemplated to include all permissible substituents of organic compounds, “permissible” being in the context of the chemical rules of valence known to those of ordinary skill in the art. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl” must still comprise the phenyl moiety and cannot be modified by substitution, in this definition, to become, e.g., a heteroaryl group such as pyridine. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

Examples of substituents include, but are not limited to, alkyl, aryl, aralkyl, cyclic alkyl, heterocycloalkyl, hydroxy, alkoxy, aryloxy, perhaloalkoxy, aralkoxy, heteroaryl, heteroaryloxy, heteroarylalkyl, heteroaralkoxy, azido, amino, halogen, alkylthio, oxo, acyl, acylalkyl, carboxy esters, carboxyl, carboxamido, nitro, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, alkoxyalkyl, perhaloalkyl, arylalkyloxyalkyl, and the like.

### EXAMPLES

The following examples are intended to illustrate certain embodiments of the present invention, but do not exemplify the full scope of the invention.

### Example 1

### Preparation and Characterization of a Triggerable Tough Hydrogel (i.e. a Composition Described Herein) (TTH)

TTHs consisting of alginate and polyacrylamide networks are crosslinked by ionic Ca²⁺ and disulfide bonds, respectively (FIGS. 1B-1C). Alginate is a linear copolymer comprised of blocks of α-L-guluronic acid, β-D-mannuronic acid, or alternating α-L-guluronic and β-D-mannuronic acids. Divalent Ca²⁺ cations can crosslink alginate by simultaneously associating with carboxylic groups in the α-L-guluronic acid blocks from different alginate chains, forming an ionically crosslinked network in water. By contrast, the polyacrylamide network can be formed by aqueous radical polymerization of acrylamide using a bifunctional monomer as the crosslinker. Since alginate and polyacrylamide networks are separately crosslinked, stimuli-responsive ionic and disulfide bonds can be incorporated making the gels susceptible to degradation by biocompatible chelators and reducing agents, TTHs can be de-crosslinked and dissolved into solution accordingly.

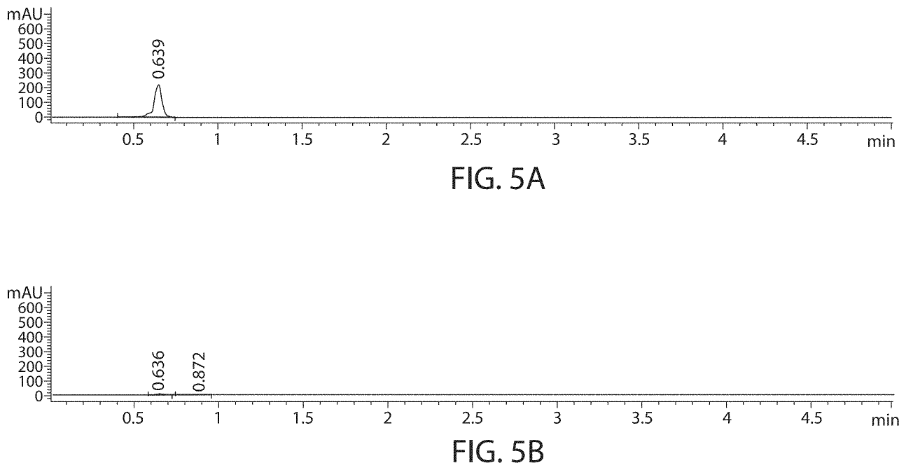

TTHs were fabricated by a simple one-step method. All ingredients needed to form the two networks were dissolved in deionized water, including sodium alginate and an ionic crosslinker of calcium sulphate for the ionically crosslinked alginate, as well as acrylamide, crosslinking monomer N,N′-bis(acryloyl)cystamine, thermo-initiator of ammonium persulphate, and polymerization accelerator of N,N,N′,N′-tetramethylethylenediamine for the disulfide crosslinked polyacrylamide. The mixture was heated to 50° C. for 1 h and then left in a humid box for 1 day. The unreacted ingredients were purified by continuous extraction with water demonstrating elimination of the acrylamide monomer (FIG. 5). Details of the synthesis and characterization of TTHs are described below.

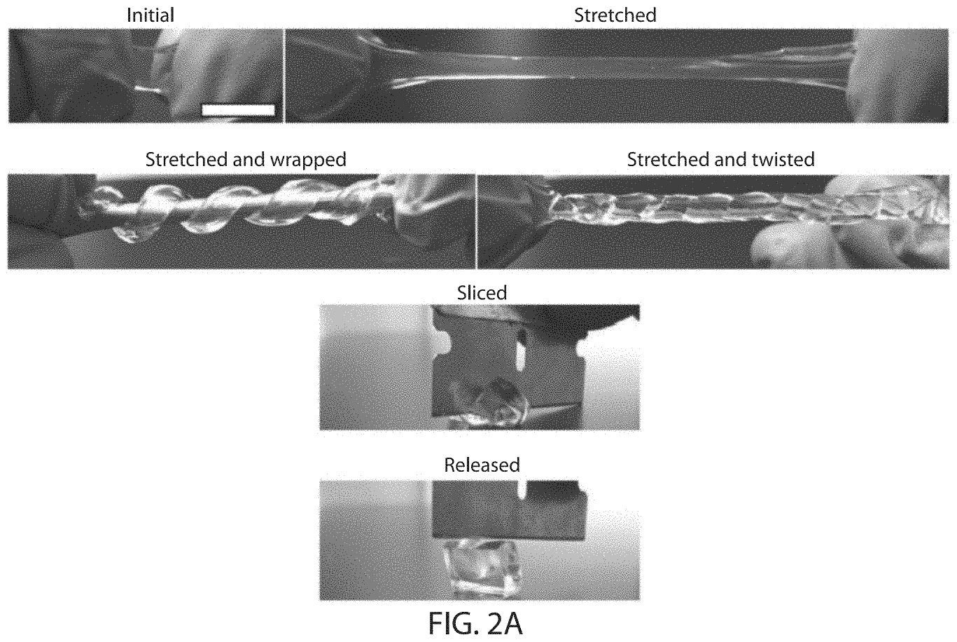

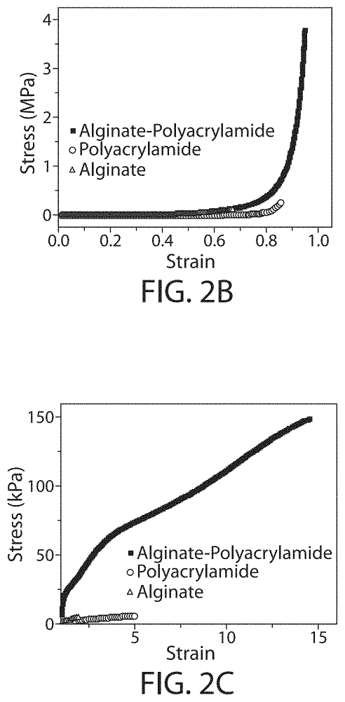

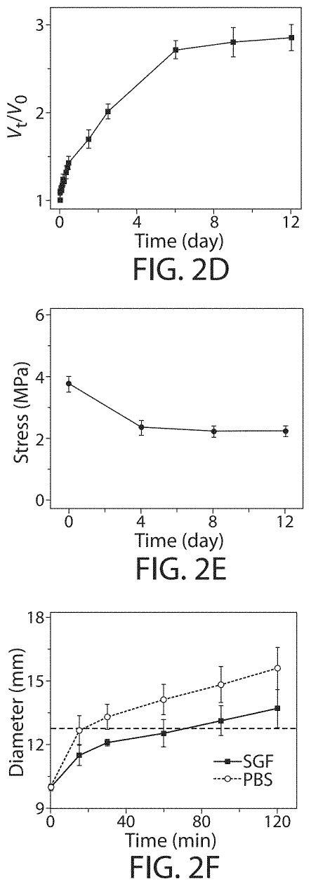

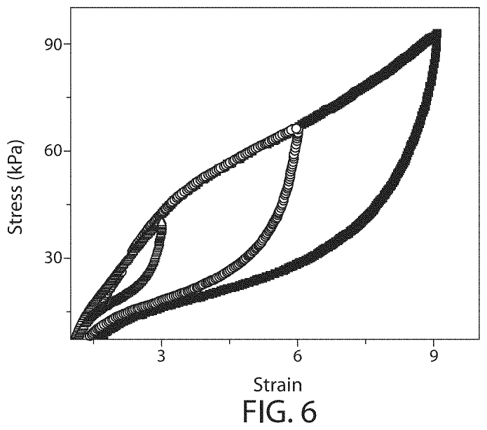

The TTH synthesized had a water content of 87%, was highly stretchable, flexible, and could not be easily cut with a blade (FIG. 2a). It achieved a maximum compressive stress of 3.78±0.26 MPa that was 14 and 32 times higher than the hydrogels composed of polyacrylamide (0.275±0.033 MPa) or alginate (0.121±0.017 MPa) alone (FIG. 2b). The tensile strength and fracture strain were, respectively, 149±11 kPa and 14.6±1.3 for the TTH, 6.2±0.8 kPa and 5.0±0.6 for the polyacrylamide gel, and 4.2±0.7 kPa and 1.9±0.3 for the alginate gel (FIG. 2c). The energy dissipation of the TTH was further tested by loading-unloading experiments, showing that the TTH dissipated energy effectively, as verified by the notable hysteresis, while the permanent deformation after unloading was negligible, as demonstrated by loading several samples to large values of stretch before unloading (FIG. 6).

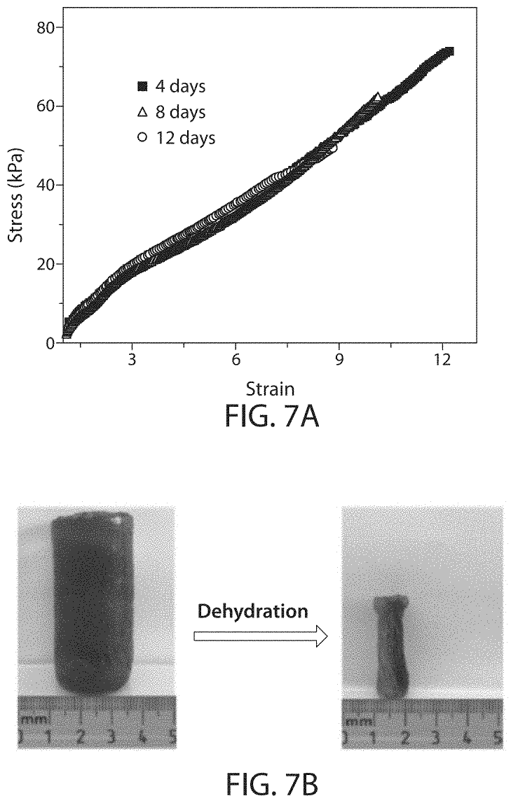

The swelling behavior and variation of mechanical properties of TTHs was next studied in simulated gastric fluid (SGF, pH=˜1.2). The TTH swelled progressively (FIG. 2d) and a plateau of volume variation (Vt/V0) of 2.7±0.15 was reached after 6 days of incubation at 37° C. with an accompanying decrease of the tensile properties at rupture. The tensile strength and fracture strain of the TTH decreased to 74.1±6.7 kPa and 12.2±1.1, 62.4±4.9 kPa and 10.1±0.8, as well as 49.2±5.7 kPa and 8.8±0.9 after incubated for 4, 8, and 12 days, respectively (FIG. 7A). The swelling also adversely affected the maximum compressive stress of the TTH, which decreased to 2.24±0.24 MPa after 4 days of incubation in SGF (FIG. 2e). After the initial decrease mainly attributed to swelling, however, the maximum compressive stress of the TTH appeared to plateau with further incubation, as confirmed by a small change from 2.21±0.18 and 2.07±0.15 MPa between 8 and 12 days of incubation, respectively. It is worth mentioning that the maximum gastric pressure in the fasted and fed states in humans is known to range from 0.01 to 0.013 MPa which is far lower than the maximum compressive stress of the TTH even following incubation periods of up to 12 days, suggesting the potential of these gels to resist gastric compression and achieve relative long-term residence in the gastric cavity.

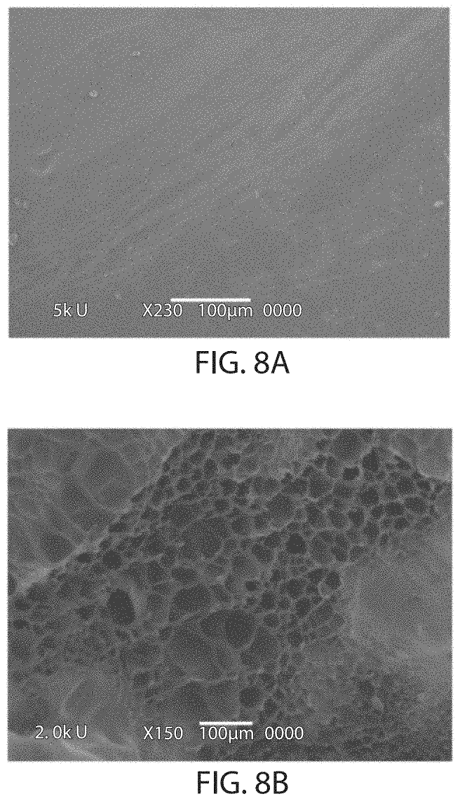

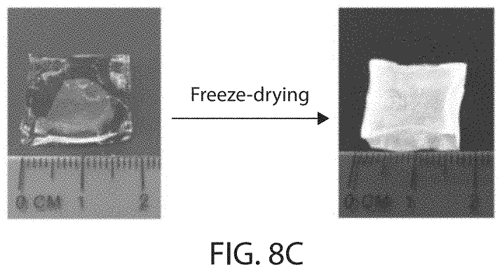

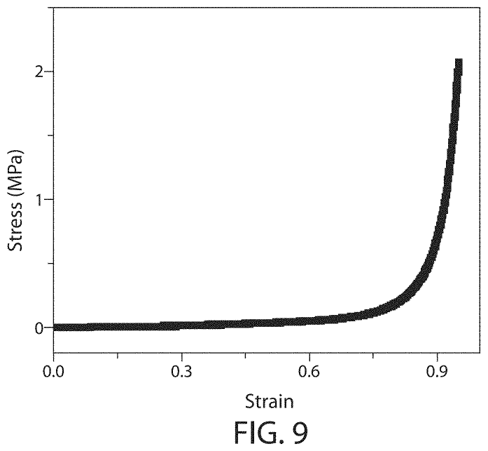

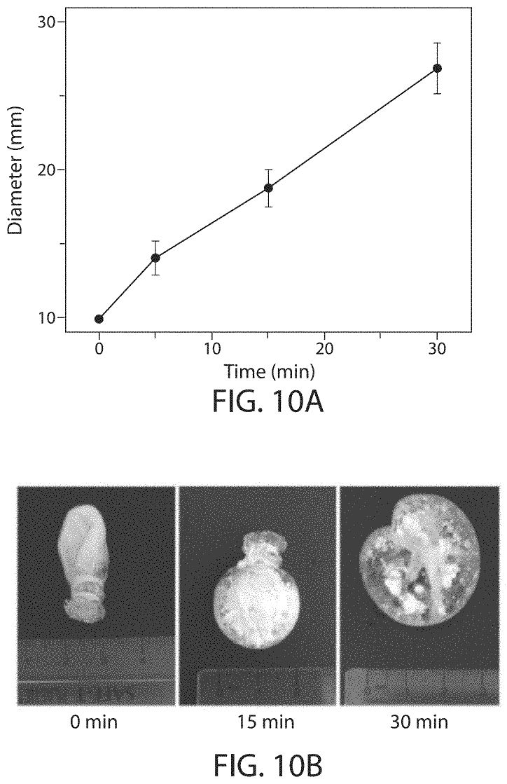

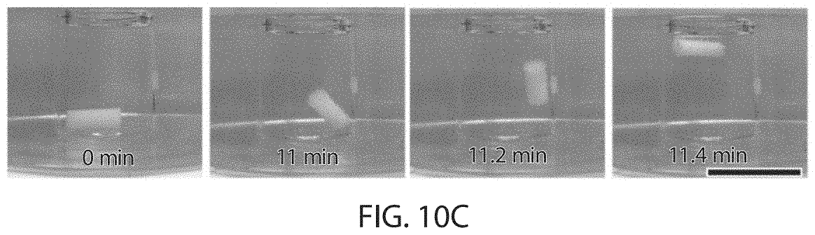

The dehydration and rehydration of TTHs was measured and found that air drying effectively dehydrated and shrunk the gel significantly (FIG. 7b). Scanning electron microscopy (SEM) images displayed a uniform structure of the dried TTH sample (FIG. 8a). As expected, the TTH could not dehydrate into a smaller size by lyophilization and a micro-porous structure was obtained for the lyophilized sample (FIG. 8b and FIG. 8c). The rehydration of TTHs in SGF was measured and found that a completely dehydrated TTH with similar dimensions to a standard 000 capsule swelled to a size greater than the diameter of the resting human pylorus (12.8±7.0 mm) within 70 minutes (FIG. 2f), which is within the 50 percentile for gastric emptying in humans. Additionally, the dehydrated TTH could swell to a size larger than the diameter of pylorus within 15 minutes in a neutral pH approximating the fed state or patients taking antacids or that can be achieved by co-administration with antacids. The enhanced swelling is attributed to the higher solubility of alginate in neutral pH than in an acidic environment. It was found that the adequately rehydrated gel demonstrated a maximum compressive stress of 2.02±0.18 MPa (FIG. 9), demonstrating the retention of toughness of TTHs after a cycle of complete dehydration and subsequent rehydration. Alternatively, a TTH-based encapsulation system encasing CaCO₃ in an initial form factor of a standard 000 capsule swelled to 27 mm within 30 minutes in SGF (FIGS. 10A-10B). Similar strategies can be applied for enabling flotation of TTHs (FIG. 10C). Given the highly stretchable and tough characteristics, various dosage forms compatible with ingestion and subsequent gastric residence through size exclusion could be developed by using TTHs.

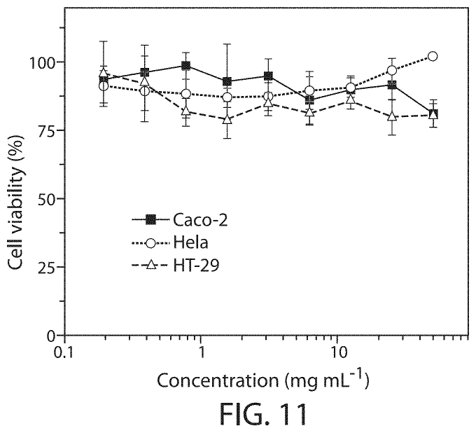

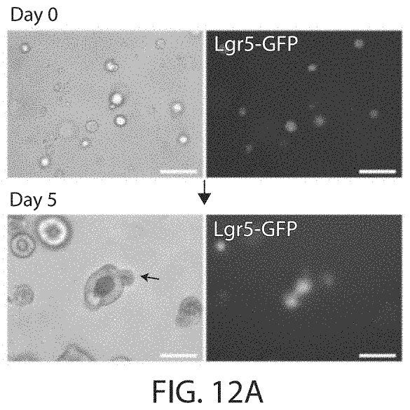

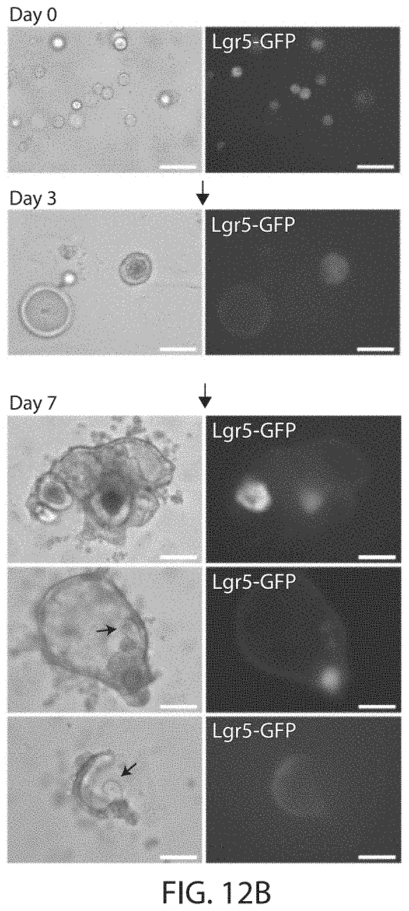

Initial biocompatibility of TTHs was evaluated through in vitro cell toxicity analysis. The gel was incubated in cell culture medium across a wide range of concentrations from 0.2 to 50 mg mL−1 at 37° C. for 24 h. The medium was then tested for its cytotoxicity on multiple cell lines, including HeLa, Caco-2 (C2BBe1 clone) and HT29-MTX-E12 (FIG. 11) No significant cytotoxicity was observed for the medium incubated with the gel in any of these cell lines at the end of a 24 h culture period. Extended cytotoxicity analysis was performed by culturing the TTHs with intestinal stem cells (ISCs) and demonstrate excellent cytocompatibility of the TTHs with mouse Lgr5+ stem cells over the course of 5 days (FIG. 12A). Furthermore, it was shown that Lgr5+ stem cells could be cultured on and within TTHs and these retained their ability of multilineage differentiation to form organoids (FIG. 12B), supporting the biocompatibility and potential application of TTHs serving as a substrate for organoid culture.

### Example 2

### Triggerable Properties of TTHs

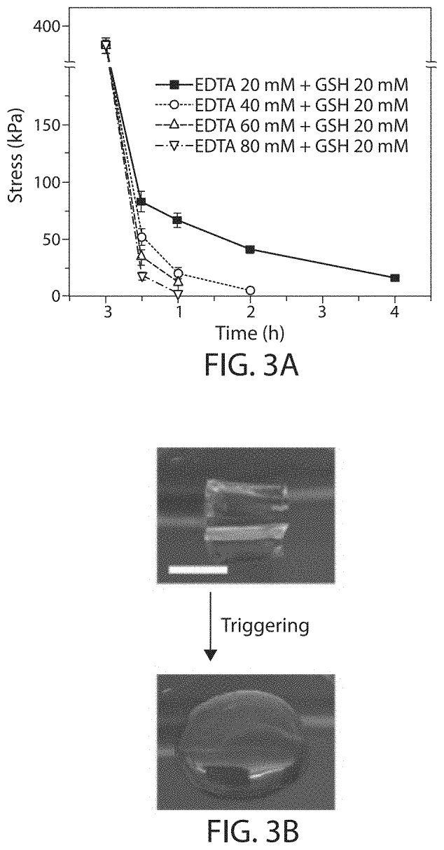

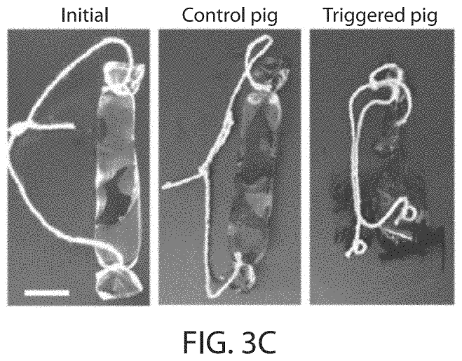

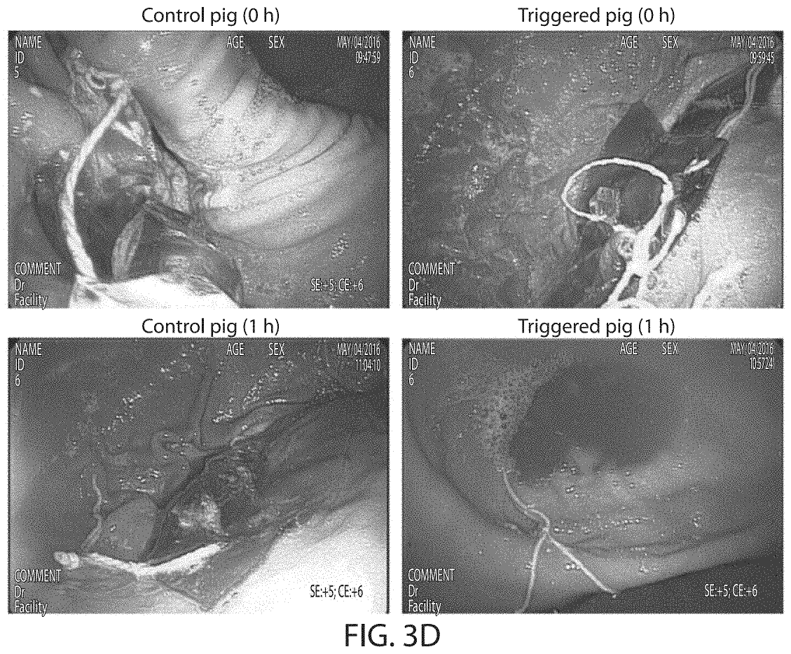

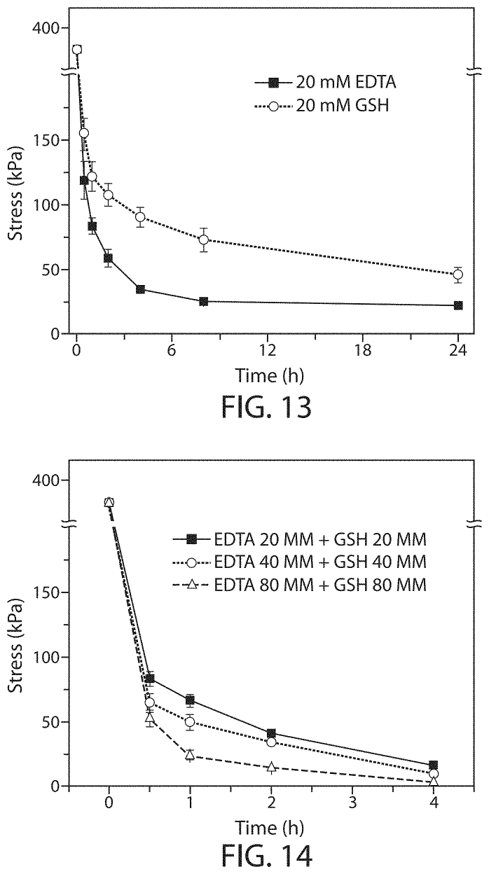

The stimuli-responsiveness of TTHs by using ethylenediaminetetraacetic acid (EDTA) and glutathione (GSH) as triggers of the Ca²⁺ ion and disulfide crosslinks was investigated. Both EDTA and GSH have been previously used in humans as treatments or supplements with oral dosages of up to 6 and 5 g daily, respectively and have been used as additives in foods. To measure the triggerable properties and potential boundaries set by EDTA and GSH found in a human diet, TTHs were incubated at 37° C. with a range of concentrations from 20 to 80 mM of EDTA and GSH well above the concentrations found in food for various time intervals, and then evaluated for compressive stress to characterize the dissolution behavior of the gels. Interestingly and supporting the selectivity of the EDTA and GSH combined triggering solution, the TTH could not be dissolved by incubation with EDTA or GSH alone even when incubation times were increased to 24 h (FIG. 13). These data support the ability to maintain a network by the remaining crosslinked single network hydrogel and demonstrate that de-crosslinking of both alginate and polyacrylamide networks are essential to dissolve the TTH. The requirement for both EDTA and GSH for triggering supports the likely sustained stability of the TTH in the presence of a normal human diet. The dissolution of the gels was accelerated by triggering with EDTA and GSH simultaneously. As shown in FIG. 3a, the compressive stress of the TTH decreased rapidly from 373±10 to 66.3±5.8, 41.3±3.9, and 16.7±1.0 kPa after 1, 2, and 4 h incubation in 20 mM of EDTA and 20 mM of GSH. When the concentration of EDTA was increased to 40 mM while the GSH was kept constant, the compressive stress of the

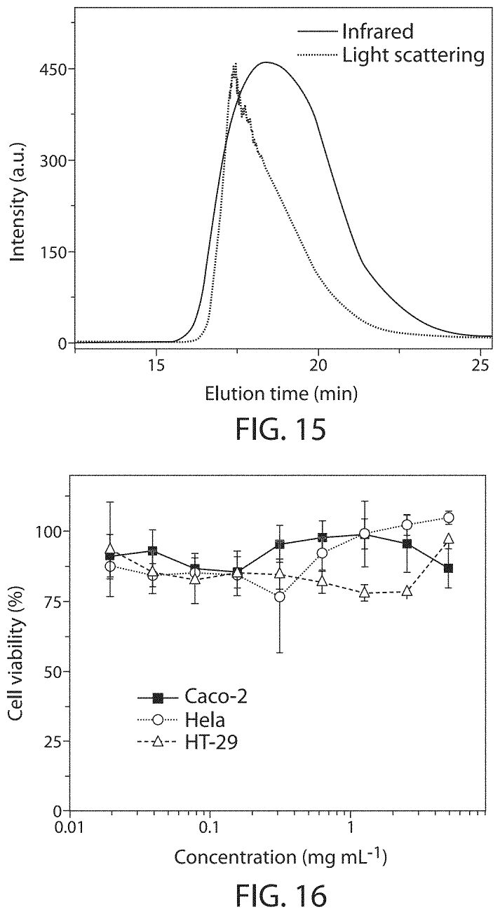

TTH reduced dramatically to 5.6±0.04 kPa and the gel dissolved after 2 h of incubation. The TTH started to dissolve into a viscous solution only after 1 h incubation with further increases of EDTA to 80 mM (FIG. 3b). In contrast, increases in GSH concentration retarded the dissolution of the TTH (FIG. 14), suggesting that the carboxyl group at the C-terminus of GSH could disturb the formation of the ionic bond between the Ca2+ and the carboxyl groups in EDTA when excessive GSH was present. Gel permeation chromatography (GPC) of the dissolved TTH demonstrated two peaks with molecular mass of ˜120 and ˜200 kDa that corresponded to the dissociated alginate and polyacrylamide chains, respectively, supporting the dissolution of the TTH into free polymers (FIG. 15). In vitro cell viability assays verified the low cytotoxicity of these dissociated free polymers against HeLa, Caco-2 and HT29 cell lines at the end of a 24 h culture with concentrations up to 5 mg mL−1 (FIG. 16).

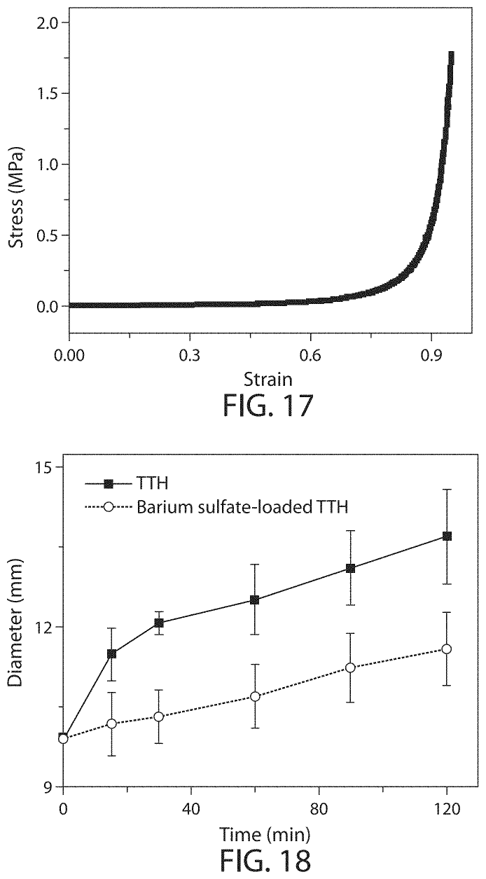

Having confirmed in vitro the superior stimuli-responsiveness of the gels, it was next tested the in vivo dissolution of TTHs by using a Yorkshire pig animal model which has been previously established for the evaluation of GI resident systems. Yorkshire pigs weighing 45-55 kg have gastric and intestinal anatomy and dimensions similar to humans46. TTH strips with dimensions 50 mm×10 mm×5 mm were introduced endoscopically into the stomach. Pigs were administered a triggering solution consisting of 0.5 L of EDTA (40 mM) and GSH (20 mM) after deployment of the TTH strips. Control samples were deployed into the stomach without the addition of the triggering solution. The TTH strips were retrieved endoscopically after 1 h in the gastric cavity. Strips retrieved from the control pigs remained intact and retained a maximum compressive stress of 1.77±0.15 MPa (FIG. 17), whereas the strips from the treated pigs dissolved into viscous solution (FIG. 3c). To further view the in situ dissolution of TTHs in stomach, large TTH sheets, in the shape of an equilateral triangle (side length, 10 mm; thickness, 3 mm) were prepared and labeled with methyl blue. These were triggered in situ with the EDTA/GSH solution and endoscopic videography was used for image capture. Endoscopic video revealed that the TTH sheets were triggered to dissolve within 1 h in the gastric cavities of the treated pigs, whereas the sheets in the control pigs remained intact (FIG. 3d). These results support that TTHs can be triggered to dissolve in vivo with biocompatible agents. Generally, uncontrolled long term (>24 h) gastric resident systems may present risks to patients including gastrointestinal mechanical obstruction and the inability to discontinue a drug in the event of developing an allergic reaction through non-invasive means. The ability to trigger the dissolution of such systems is therefore useful for safe clinical implementation. The need for triggering is further amplified in resource constrained settings where healthcare interventions like endoscopy and surgery may be largely limited and where the inability to remove such systems could manifest in significant morbidity and mortality.

### Example 3

### Gastric Retentive Drug Delivery of TTHs

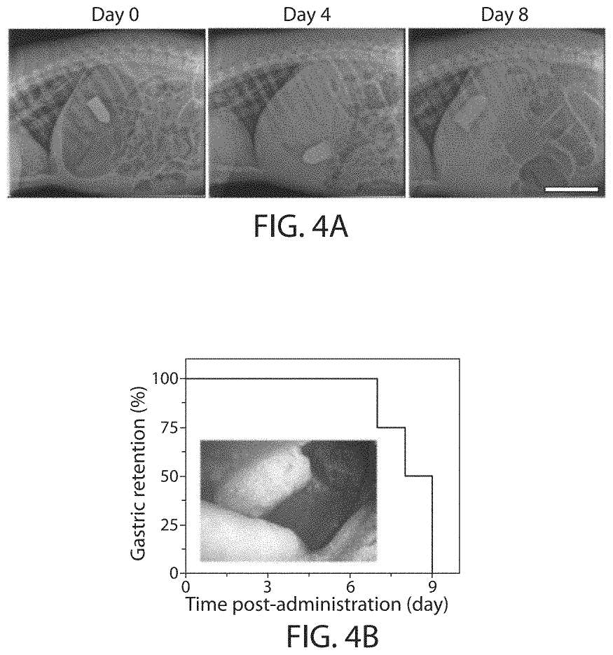

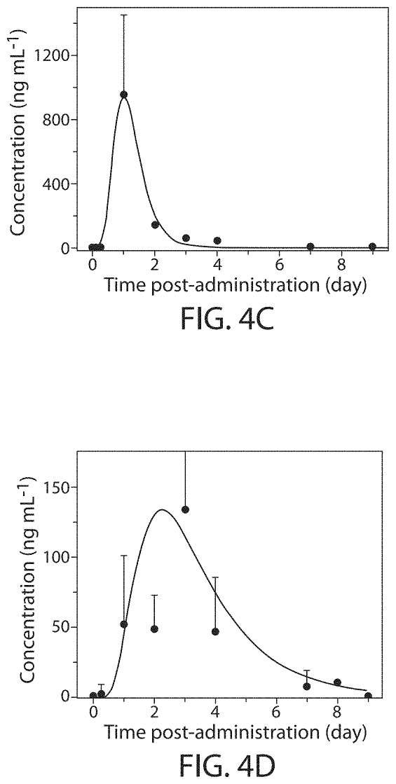

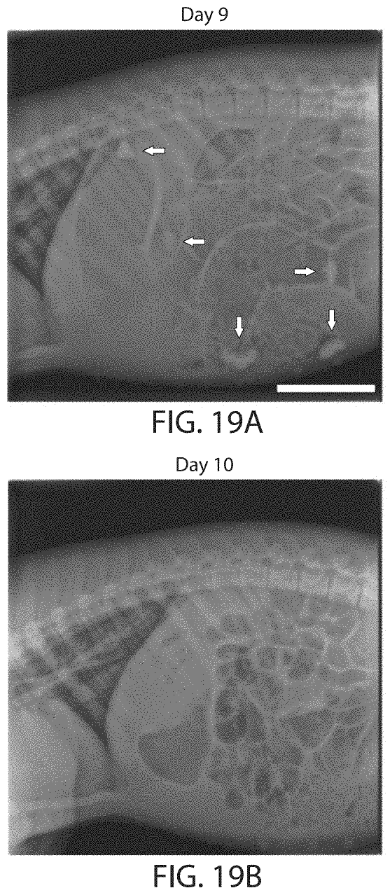

To evaluate the mechanical integrity of TTHs and their potential application as triggerable biomedical materials in gastric resident systems, TTH prototypic gastric resident dosage forms were fabricated. These were evaluated for their gastric residence and integrity in Yorkshire pigs. To evaluate the gastric retention and in vivo integrity of TTHs, radiopaque capsule-like TTH dosage forms with volumes of 22 mL (diameter, 2.8 cm; total length, 5 cm) were designed and prepared by mixing barium sulfate with the pre-gel solution immediately prior to polymerization. It was noted that the significant load of barium sulfate required for radiographic visualization (20 wt %) manifested in slower swelling characteristics than the TTHs (FIG. 18). Barium sulfate-containing TTHs in their hydrated states were used which enabled the retention by virtue of the size of the gel administered and radiographic visualization by virtue of their barium content. Four individual experiments in four different pigs were performed and radiographs were taken approximately every 48-72 h to monitor the integrity of the dosage form, its anatomic location and any evidence of GI obstruction. Intact prototype TTH systems were observed to achieve gastric residence of 7 to 9 days (FIG. 4a,b). TTHs remained stable in vitro in SGF (>12 days) though in vivo breakage of TTHs was observed earlier than this may be due, in some cases, to the compressive stress associated with gastric motility, and potential de-crosslinking of alginate network by exchange reactions with monovalent cations in the GI environment. Meanwhile, the disulfide bonds in polyacrylamide network may be, in some cases, reduced by protein or peptide associated thiols though low molecular weight thiols, glutathione and cysteine are only present at a low level or even absent in human gastric fluid. No intact devices were visualized outside of the gastric cavity, supporting that device breakage (i.e. disassociation) first occurred in the stomach enabling their eventual passage out of the stomach. Once device breakage occurred, the resulting fragments were visualized in the intestines without evidence of intestinal obstruction (FIGS. 19A-19B). Throughout the experiments the animals were found to have normal eating and stooling patterns and did not exhibit any signs of GI obstruction, either clinically or radiographically.

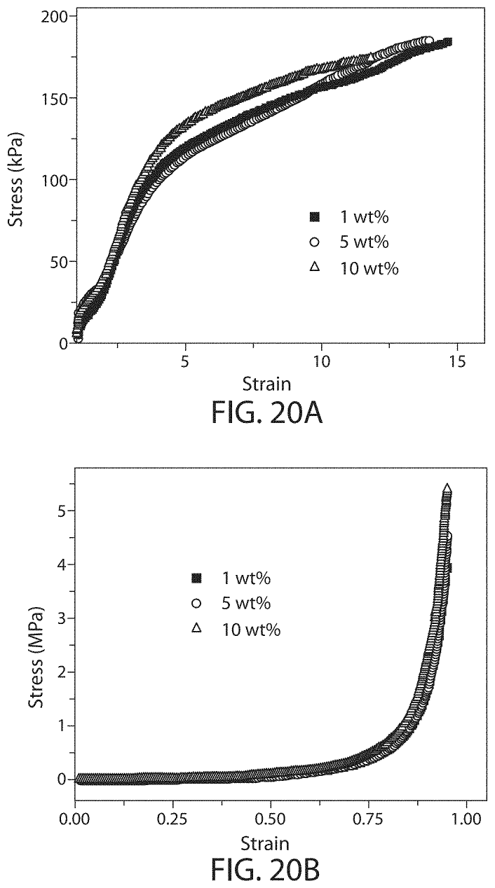

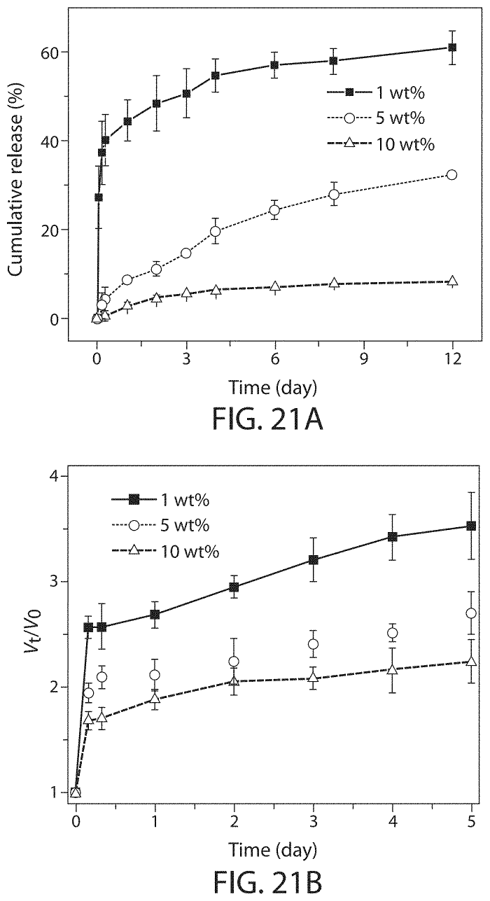

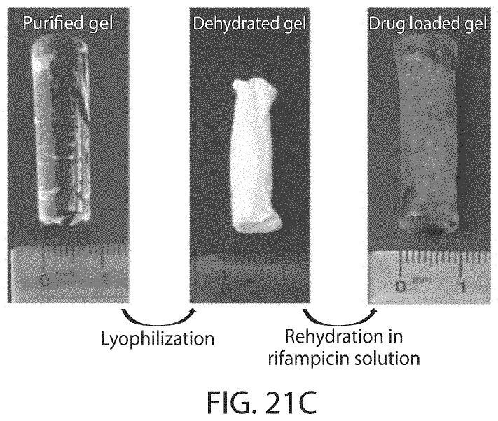

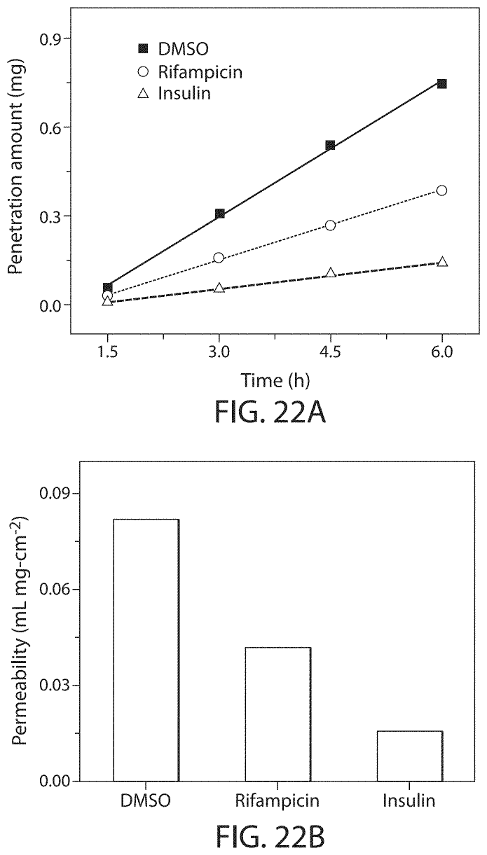

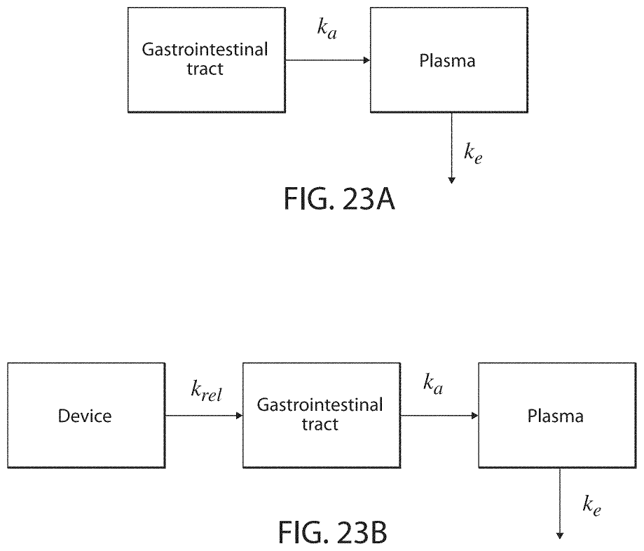

Medication non-adherence is a major challenge for the treatment of malaria and having the capacity to deliver drugs in a single administration event has the potential to not only enhance cure rates in acute malaria but also decrease resistance rates. To demonstrate a potential application of this system a gastric resident TTH dosage form containing lumefantrine, a hydrophobic antimalarial drug, was selected to study the drug loading and release from the TTH material. The lumefantrine-loaded TTHs were similarly fabricated by mixing drug powder with the pre-polymerization solution just before gelation. The degree of drug loading was easily controlled by adjusting the feed ratio of drug. The maximum compressive stress of the gel increased from 3.91±0.31 to 5.43±0.61 MPa with the increase of drug loading from 1 to 10 wt %, whereas the fracture strain decreased from 14.7±1.3 to 11.9±1.5 and the tensile strength remained around 180±20 kPa (FIGS. 20A-20B). In vitro release kinetics of the lumefantrine dosage forms were characterized under predetermined sink conditions and the results showed that the release of lumefantrine could be controlled by tuning the drug loading. In vitro characterization of the cumulative release of lumefantrine after 12 day incubation in SGF increased from 8.3±0.17% to 61±3.7% with the decrease of drug loading from 10 wt % to 1 wt %, suggesting the diffusion of drug was decreased as a function of reduced swelling of the TTH associated with the increase in hydrophobicity of the gels from the higher lumefantrine load (FIGS. 21A-21B). A first order rate equation was fit to describe the rate of drug release, and the release rate constants from gels loaded with 1%, 5% and 10% drug were found to be 11.1 day-1, 0.2 day-1 and 0.36 day-1 respectively. It was noted that the post-polymerization purification affected the drug loading of TTHs prepared by mixing drug powder with the pre-polymerization solution. An alternative strategy was demonstrated to avoid drug loss during the preparation of drug-loaded TTHs by using post-polymerization encapsulation. As shown in FIG. 21c, the purified TTH was first prepared, then lyophilized and subsequently rehydrated the TTH in the aqueous solution of drugs. Additionally, to evaluate the potential delivery of a range of molecules, transport of model molecules across a range of molecular weights was evaluated through TTHs. Specifically, insulin, rifampicin and dimethyl sulfoxide were observed to transport efficiently through the TTH and showed size-dependent permeability that increased from 0.016, 0.042 to 0.082 mL h·cm−2 with the decrease of molecular weight from 5808, 823 to 78 Da (FIGS. 22A-22B). To evaluate the release kinetics from TTH in vivo, lumefantrine-loaded TTH systems in the same dimensions and shape to the TTH system used for the gastric retention and integrity studies were prepared. The pharmacokinetic studies were carried out by single administration of one drug-loaded TTH device containing 960 mg of lumefantrine per pig. The in vivo pharmacokinetics were significantly extended when administered in the form of TTH as compared to the unformulated free drug control (FIG. 4c,d). After a single administration of free lumefantrine, the drug was rapidly cleared from blood with a rapid terminal elimination phase (FIGS. 23A-23B). In contrast, a relative constant blood drug concentration remained up to 4 days after a single administration of the lumefantrine-loaded TTH device, supporting the potential for multi-day dosing using the TTH drug delivery system. A pharmacokinetic model described by first order rate equations was fit to the data. The absorption rate constant for both formulations was 1.17 day−1. The rate constant for drug release in vivo was estimated to be 0.68 day-1, which is ˜3-fold higher than the in vitro release rate constant. This may be because in vitro tests do not account for food effects and other gastric secretions, which may significantly affect drug release. The elimination rate constants for the free drug was estimated to be 1.17 day-1 and that apparent elimination rate constant of the drug delivered in TTH was 0.68 day-1 indicating delayed elimination.

In summary, a novel family of triggerable tough hydrogels were developed and demonstrate their capacity for significant dehydration and rehydration. Their capacity to be triggered to dissolve with the application of biocompatible triggers was demonstrated. TTHs were evaluated for their stability and mechanical integrity in a large animal model. A potential application in drug delivery was also demonstrated with an extended release system for lumefantrine. Pre-clinical studies will be required to translate these systems for human application including further safety studies and stress testing in other large animal models. In sum, the TTHs described herein present three important points of novelty from the hydrogel perspective: exceptional mechanical properties: that can withstand in vivo gastric forces and achieve long-term residence in the stomach of a large mammal; remarkable triggerable properties: capable of on-demand dissolution; TTHs can be drug loaded and provide controlled drug release. It is believed that, in one set of embodiments, this combination of features makes TTHs uniquely attractive for the development of advanced gastric dosage forms for prolonged drug delivery, ingestible electronics, and bariatric applications.

### Example 4

### Methods for Compositions and Experiments Described in Examples 1-3

Materials. Acrylamide (A9099, ≥99%), N,N′-bis(acryloyl)cystamine (A4929), ammonium persulfate (A3678, ≥98%), N,N,N′,N′-tetramethylethylenediamine (T9281, 99%), sodium alginate (A2033, medium viscosity), calcium sulfate (C3771, ≥99%), methyl blue (M6900), barium sulfate (11844), L-glutathione reduced (GSH, ≥98%), ethylenediaminetetraacetic acid (EDTA, ≥99%), dimethyl sulfoxide (DMSO, D8418), sodium bicarbonate (NaHCO3, S5761), calcium carbonate (CaCO3, 99%), and phosphate buffered saline (PBS, pH 7.4) were available commercially from Sigma-Aldrich and used as received unless otherwise noted. Insulin was kindly provided by Novo Nordisk and labeled by Alexa-Fluor® 488. Lumefantrine and rifampicin were purchased from Hangzhou Hysen Pharma CO., LTD in China. Nanopure water (18 MS/cm) was acquired by means of a Milli-Q water filtration system, Millipore (St. Charles). Simulated gastric fluid (SGF, pH ˜1.2) was made by dissolving 2 g NaCl and 8.3 mL concentrated HCl in nanopure water and adjusting to 1,000 mL.

Mechanical characterization. The mechanical characterization in tension and compression was performed on an Instron testing machine according to ASTM standards D638 (tension) and D575 (compression). For tensile measurement, specimens were loaded into the grips with a 50 N load cell and the gauge length measured using a digital micrometer. Displacement was applied to the specimen at a rate of 0.15 mm s−1 until samples ruptured. For compression measurement, specimens were placed into a constrained loading compression jig with a 500 N load cell and the gauge length measured using a digital micrometer. Displacement was applied to the specimen at a rate of 0.05 mm s−1 until reaching 95% compressive strain. Force was converted into pressure (F/A) and displacement into strain (ΔL/L).

High performance liquid chromatography (HPLC). HPLC measurement was carried out on an Agilent 1260 Infinity HPLC system equipped with a quaternary pump, autosampler, thermostat, control module, and diode array detector (DAD). The output signal was monitored and processed using the ChemStation® software. Chromatographic separation was carried out on a 50 mm×4.6 mm EC-C18 Agilent Poroshell 120 analytical column with 2.7 μm spherical particles, maintained at 40° C. The optimized mobile phase consisted of acetonitrile, methanol, and buffer (pH 3.5 adjusted with 0.1% formic acid) (72:20:8, v/v) at flow rate of 0.5 mL min−1 over a 10 min run time. The injection volume was 4 μL, and the UV detection wavelength of 254 nm was selected.

Liquid chromatography tandem-mass spectrometry (LC-MS/NIS). UPLC separation was conducted on a Waters UPLC aligned with a Waters Xevo-TQ-SMS mass spectrometer (Waters Ltd., UK). MassLynx 4.1 software was used for data acquisition and analysis. Liquid chromatography separation was performed on an Acquity UPLC CSH C18 (50×2.1 mm, 1.7 μm particle size) at 50° C. The mobile phase consisted of acetonitrile, 0.1% formic acid, and 10 mM ammonium formate was flowed at a rate of 0.6 mL min−1 using a time and solvent gradient composition. The initial gradient (100%) was followed by a linear gradient (20%) over 0.25 min. Over the next 1.25 min the gradient was brought to 0% and held for 0.5 min and finally brought back to the initial gradient of 100% over 0.25 min and held until the end of the run for column equilibration. The total run time was 4 min and sample injection volume was 2.5 μL. The mass spectrometer was operated in the multiple reaction-monitoring (MRM) mode. Sample introduction and ionization was ESI in the positive ion mode. Stock solutions of lumefantrine and an internal standard artemisinin were prepared in methanol at a concentration of 500 μg mL−1. A ten-point calibration curve was prepared ranging from 2.5-2500 ng mL−1. Quality control samples were prepared in a similar procedure using an independent stock solution at three concentrations (2.5, 25 and 250 ng mL−1).

200 μL of internal standard 250 ng mL−1 was added to 100 μL of sample solution to cause precipitation. Samples were vortexed and sonicated for 10 min and then placed in a centrifuge for 10 min. 200 μL of solution was pipetted into a 96-well plate containing 200 μL of water. Finally, 2.5 μL was injected into the UPLC-ESI-MS system for analysis.

Scanning electron microscope (SEM). Surface morphology of the dehydrated gels was observed using the JEOL 5600LV SEM. For visualization under SEM, samples were fixed to aluminum stubs with double-sided adhesive carbon conductive tape and subsequently sputter-coated with carbon using a Hummer 6.2 Sputter System.

Gel permeation chromatography (GPC). Aqueous GPC was conducted on a Viscotek system (Malvern) equipped with an isocratic pump Viscotek VE 1122 solvent delivery system, TDA 305 triple detector array, and 3 TSK Gel GMPWxL column with guard column. The system was equilibrated at 30° C. in pre-filtered water containing 0.05 M NaNO3 with the flow rate set to 1 mL min−1. Polymer solutions were prepared at a concentration of about 0.5˜5 mg mL−1 and an injection volume of 200 μL was used. Data collection and analysis were performed with ChemStation for LC (Agilent) and OmniSEC v. 4,6,1,354 software (Malvern). The system was calibrated with poly(ethylene oxide) standards (Sigma) ranging from 400 to 511,000 Da (Mp).

Preparation of TTHs. TTHs were prepared by a one-pot synthetic method. Typically, acrylamide (3.60 g, 50.6 mmol), N,N′-bis(acryloyl)cystamine (13.2 mg, 0.051 mmol), ammonium persulfate (57.8 mg, 0.253 mmol) and sodium alginate (600 mg) were dissolved into 30 mL nanopure water. N,N,N′,N′-tetramethylethylenediamine (29.4 mg, 0.253 mmol) and calcium sulfate (120 mg, 0.697 mmol) were added after a homogeneous solution was obtained. Calcium sulfate was added as a suspension into the reaction mixture because of its limited water solubility caused by its low dissociation constant. Although the association of Ca2+ with the carboxyl groups in alginate could accelerate the dissolution of calcium sulfate, the complete dissolution took place overnight. Thus the reaction mixture was presented as a free solution before subjecting it to polymerization even after all the ingredients were added. The solution was carefully degassed and then quickly poured into standard dumbbell die (ASTM D-638) moulds.

The gel was crosslinked by heating to 50° C. for 1 h, then sitting in a humid box at room temperature for another 24 h to stabilize the reaction. Afterwards, the resulted TTHs were subjected to mechanical characterization. To prepare the TTH membrane for permeability measurement, the pre-gel solution was poured into a glass mould covered with a 3-mm-thick glass plate. To prepare a TTH-based floating system, CaCO₃ powder (5 wt %) was added into the reaction mixture just before polymerization. For in vivo dissolution study, the TTH membrane was labeled with methyl blue by adding a drop of dye solution onto the top of the TTH membrane and then covered by a glass plate and further incubated overnight. To prepare radiopaque-labeled capsule-like TTHs, 20 mL pre-gel solution containing barium sulfate (20 wt %) was added into a 50 mL CORNING CentriStar™ tube immediately prior to polymerization. The drug-loaded TTHs were similarly fabricated by mixing lumefantrine powder with the prepolymerization solution just before gelation, and the degree of drug loading was easily controlled from 1 to 10 wt % by adjusting the feed ratio of drug. To prepare water soluble drug-loaded TTHs, the purified TTH was lyophilized and subsequently rehydrated in the aqueous solution of rifampicin (a water soluble antibiotic).

Purification of TTHs. To measure the unreacted ingredients in TTHs, the resulted gel was cut into 1-2 mm pieces and sonicated in 10 volumes of water for 30 minutes. The mixture was further incubated at 37° C. for 24 h on a shaker plate at 250 r.p.m. After the addition of a certain volume of acetonitrile, the mixture was centrifuged and the supernate was analyzed by HPLC. To purify the TTH, the obtained gel was extensively extracted with 4×1000 mL water for 24 h. The same procedure described above was performed to measure the unreacted ingredients in the purified TTH.

Swelling and stability of TTHs in SGF. The swelling and stability of TTHs were measured by incubating TTH samples in SGF at 37° C. and subsequent measuring the volume as well as the maximum compressive stress. Typically, the cylindrical TTH samples (diameter, 6.2 mm; length, 12 mm) were prepared by carrying out the gelation reaction in a 3.5 mL VWR glass vial. The obtained gels were submerged in 50 mL GSH in a Corning CentriStar™ tube and then incubated at 37° C. on a shaker plate at 250 r.p.m. After predetermined time intervals, the size of the samples was measured by using a digital micrometer and compared with initial volumes. Meanwhile, the TTH samples were also subjected to compression measurement. Three replicates were conducted for each TTH sample.

Dehydration and rehydration of TTHs. The dehydration of TTHs was measured by incubating TTH samples in air at 37° C. Typically, the cylindrical TTH samples (diameter, 6.2 mm; length, 12 mm) were placed in the oven set at 37° C. and the size of the samples after predetermined incubation intervals was measured by using a digital micrometer and compared with initial volumes. For rehydration measurement, the dehydrated gel samples were submerged in 50 mL SGF in a Corning CentriStar™ tube and incubated at 37° C. on a shaker plate at 250 r.p.m. After different time intervals, the size of the samples was measured and compared with initial volumes. Three replicates were conducted for each TTH sample. In a control experiment, TTH samples were frozen at −20° C. and subsequently dried by lyophilization.

Dissolution of TTHs with triggers. The dissolution of TTHs was studied by using EDTA and GSH as triggers. Typically, the TTH were cut into 1 cm3 sized cubes and submerged in 10 mL PBS (pH 7.4) containing EDTA and GSH with a range of concentrations from 20 to 80 mM in a 20 mL VWR glass vial. Three replicates for each time point and condition were incubated at 37° C. on a shaker plate at 250 r.p.m. At each time point, the TTH cubes were subjected to compression measurement. The TTH cubes incubated in either 20 mM of EDTA or GSH were used as controls. To demonstrate the complete dissolution of TTHs into free polymer chains, the triggered solutions were filtered by a 0.2 μm filter and subsequently injected into GPC. For cytotoxicity assay of the dissociated polymers, the triggered solutions were transferred to dialysis tubes (MWCO, 10 kDa), then dialyzed against pure water for three days to remove EDTA and GSH, and finally dried by lyophilization.

Permeability measurement. The measurement of permeability of TTHs was carried out on a Franz diffusion cell using a TTH membrane (thickness, 3 mm). 2 mL PBS (pH 7.4) containing 1 mg mL−1 DMSO, rifampicin, or insulin was added into the donor compartment of the cell and 12 mL fresh PBS was placed in the acceptor compartment of the cell. At each time point, 0.4 mL was sampled from the acceptor compartment and 0.4 mL fresh PBS was supplemented through the sampling port of the cell. The concentration of DMSO and rifampicin of the samples was recorded on a Perkin-Elmer Lambda ultraviolet-visible (UV-vis) spectrometer, and the UV absorbance calibration curve of DMSO in a range from 6.25 to 100 μg mL−1 or rifampicin in a range from 1.56 to 100 μg mL−1 with a correlation coefficient >99.9% was used to determine the concentration. The content of Alexa Fluor 488 labeled insulin was measured on an Infinite® M200Pro (Tecan) reader (excitation, 490 nm; emission, 540 nm).

In vitro drug release. Individual 50 mg TTH cubes with lumefantrine content of 1 wt %, 5 wt %, or 10 wt % were used for long-term release studies. Typically, the TTH cubes were submerged in 2 mL SGF in a 15 mL VWR centrifuge tube and then incubated at 37° C. on a shaker plate at 250 r.p.m. At each time point, the release medium was replaced by 2 mL fresh SGF and then frozen at −80° C. until analysis. The release study was carried out for up to 12 days and the total drug release was measured by HPLC using a linear standard curve of lumefantrine with a range of concentration from 0.005 to 50 μg mL−1 and a correlation coefficient>99.9%.

Cytotoxicity assay. The cytotoxicity assay of the dissociated polymers was conducted by adding the polymers directly into the culture medium with a range of concentrations from 0.02 to 5 mg mL−1. For the TTH, the gel was incubated in the culture medium with a range of dosage from 0.2 to 50 mg mL−1 at 37° C. for 24 h. The obtained medium was then tested for its toxicity towards cells. Cell lines were purchased from ATCC and Public Health England for these experiments. To avoid cross contamination, expanded cells were stored in individual containers. Regular mycoplasma evaluations were performed of the cell culture environment and the cell lines to ensure the absence of mycoplasma contamination. Cytotoxicity was tested on HeLa, C2BBe1 (ATCC) and HT29-MTX-E12 cells (Public Health England) by seeding them each in a 96-well plate at a density of 10,000 cells well-1. HeLa and HT29-MTX-E12 cells were cultured in 100 μL Dulbecco's Modified Eagle Medium (DMEM) (Life Technologies) containing 1% non-essential amino acids, 10% fetal bovine serum (FBS) and 1% penicillin streptomycin solution (Life Technologies) per well. C2BBe1 cells were cultured in the same medium, but the 1% non-essential amino acids were replaced with 1% human insulin-transferrin-selenium (Life Technologies). Cells were kept in culture for three days before replacing the medium with 100 μL of the pre-prepared solutions. After 24 h, these solutions were replaced with 100 μL untreated media and cytotoxicity was quantified by adding 10 μL alamarBlue reagent (Life Technologies) to each well. The contents were mixed and then allowed to incubate at 37° C. for 1 h. Fluorescence was recorded on an Infinite® M200Pro (Tecan) with excitation at 560 nm and emission at 590 nm. A positive control was provided by lysing cells with ethanol and cells that were not subject to any polymer-treated media provided a negative control. Cell viability was calculated by the following equation: Cell viability (%)=100×[Absorbance(sample)−Absorbance(positive control)]/[Absorbance(negative control)−Absorbance(positive control)].

Stem cell culture. Cell culture media (Advanced DMEM/F12 with N2, B27, and N-acetylcysteine) containing growth factors (EGF, Noggin, R-Spondin 1) and small molecules (CHIR99021 and VPA) were used for stem cell culture. All animal experiments were performed in accordance with protocols approved by the Committee on Animal Care at MIT. Single mouse Lgr5-GFP intestinal stem cells were isolated from Lgr5-EGFP-IRES-CreERT2 mice (Jackson Labs) as described previously 50. The isolated single Lgr5-GFP stem cells were cultured in Matrigel for 2 days to form stem cell colonies before use. To evaluate the cytobiocompatibility of TTHs against stem cells, the TTH dishes (thickness: 1 mm, diameter: 8 mm) were cultured directly with Lgr5-GFP stem cell colonies in a 24-well plate for 5 days. To test the ability of TTHs serving as a substrate for organoid culture, Lgr5-GFP stem cell colonies were mixed with Matrigel and then placed on the lyophilized TTH dishes in a 24-well plate. The plate was either placed in a 37° C. incubator directly (for cell culture on the TTHs), or further incubated for 30 min on ice and subsequently centrifuged at 300 g for 2 min before placed in a 37° C. incubator (for cell culture within the TTHs). Cells were cultured in stem cell culture media for 3 days before switching to organoid culture media (by removing CHIR99021 and VPA to permit spontaneous differentiation of the stem cells) and further cultured for another 4 days.

In vivo studies. All animal experiments were performed in accordance with protocols approved by the Committee on Animal Care at MIT. A large animal model, 45-55 kg Yorkshire pigs, was chosen as its gastric anatomy similar to humans and is widely used in evaluating devices in the GI space. Pigs were sedated with Telazol (tiletamine/zolazepam) 5 mg kg−1, xylazine 2 mg kg−1, and atropine 0.05 mg kg−1, and/or isoflurane (1˜3% inhaled), and an endoscopic overtube (US Endoscopy) was placed in the esophagus under endoscopic visual guidance during esophageal intubation. To evaluate the TTH for its ability to be triggered to dissolve into solution with biocompatible agents, the retrievable TTH strips and methyl blue-labeled large TTH sheets were administered via the overtube into the stomach. PBS (0.5 L) containing EDTA (40 mM), GSH (20 mM), and NaHCO3 (60 mM) was administered via the overtube after the gastric placement of the TTH samples. Intra-gastric endoscopy videography was used for image capture of the dissolution of the TTH sheets. The TTH strips were retrieved endoscopically after 1 h in the gastric cavity. Pigs that were not administered EDTA/GSH and therefore the strips and sheets were only exposed to gastric fluid were used as control experiments. To assess TTHs for the ability to achieve gastric retention, radiopaque barium sulfate-labeled capsule-like TTHs were administered via the overtube into the gastric cavity (one TTH device per pig). Radiographs were performed every 48-72 h to monitor the integrity and transit of the devices as well as any radiographic evidence of bowel obstruction or perforation. In vivo drug release experiments were performed with dosage forms (one drug-loaded TTH device in their hydrated states containing 960 mg of lumefantrine per pig) in the same dimensions and shape to the barium sulfate-loaded TTH device. Blood samples were obtained via cannulation of an external mammary vein on the ventral surface of the pig at indicated time points, most often time 0 min (prior to administration of the dosage form), 5 min, 15 min, 30 min, 2 hours, 6 hours, and then daily for a minimum of 5 days and then three times per week. During the evaluation of the TTH systems for gastric residence and drug delivery the animals were monitored twice daily for any signs of abnormal feeding and stooling patterns. Additionally the animals were monitored clinically for any evidence of gastrointestinal obstruction as well as radiographically every 48-72 hours for evidence of obstruction and/or perforation.

Modeling pharmacokinetic data To determine the elimination rate constant and half-life of the free lumefantrine, a one compartment oral absorption model was fit to the pharmacokinetic data. The model is shown in FIG. 23A. Using this model, an equation was derived to describe the plasma concentration time profile:

$${C}_{p} = \frac{Dose {k}_{a}}{V ( {k}_{a} - {k}_{e} )} [ {e}^{- {k}_{e} t} - {e}^{- {k}_{a} t} ]$$

To describe the pharmacokinetic profile obtained from the lumefantrine-loaded TTH device, a pharmacokinetic model described in FIG. 23B was used. An equation was obtained to describe the plasma concentration:

$${C}_{p} = \frac{D o s e {k}_{rel} {k}_{a}}{V} [ \frac{{e}^{- {k}_{rel} t}}{( {k}_{a} - {k}_{rel} ) ( {k}_{e} - {k}_{rel} )} - ( \frac{1}{{k}_{a} - {k}_{e}} ) ( \frac{{e}^{- {k}_{e} t}}{{k}_{rel} - {k}_{e}} - \frac{{e}^{- {k}_{a} t}}{{k}_{rel} - {k}_{α}} ) ]$$

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States

Patent Office Manual of Patent Examining Procedures, Section 2111.03.

1. A composition, comprising:
a polymer network comprising first and second interpenetrating polymers; and
a first cross-link moiety associated with the first polymer, configured to disassociate upon interaction with a first reagent, wherein the composition has a first configuration having an average cross-sectional dimension of less than or equal to 30 cm, and
wherein the composition has a second configuration, different than the first configuration such that the composition is retained at a location internal to a subject for greater than or equal to 24 hours in the second configuration.

2. A composition as in claim 1, comprising a second cross-link moiety associated with the second polymer, configured to disassociate upon interaction with a second reagent different than the first reagent.

3. A composition as in claim 1, wherein the composition in the second configuration comprises greater than or equal to 70 wt % fluid versus the total weight of the composition.

4. A composition as in claim 1, wherein the first cross-link moiety comprises an ionic bond.

5. A composition as in claim 1, wherein the first reagent comprises a chelator.

6. A composition as in claim 1, wherein the first reagent dissociates the ionic bond.

7. A composition as in claim 1, wherein the second cross-link moiety comprises a disulfide bond.

8. A composition as in claim 1, wherein the second reagent comprises a reducing agent.

9. A composition as in claim 1, wherein the second reagent disassociates the disulfide bond.

10. A composition as in claim 1, wherein the ionic bond is a polyvalent cation ionic bond.

11. A composition as in claim 1, wherein the ionic bond comprises calcium.

12. A composition as in claim 1, wherein the first polymer comprises alginate.

13. A composition as in claim 1, wherein the second polymer comprises polyacrylamide.

14. A composition as in claim 1, comprising an active pharmaceutical ingredient associated with the polymer network.

15. A composition as in claim 1, wherein the second configuration comprises swelling the polymer network.

16. A composition as in claim 1, wherein the composition has a maximum compressive stress of greater than or equal to 1 MPa and less than or equal to 10 MPa.

17. A composition as in claim 1, wherein the composition has a tensile strength of greater than or equal to 40 kPa and less than or equal to 200 kPa.

18. A composition as in claim 1, wherein the composition has a fracture strain of greater than or equal to 5% and less than or equal to 20%.

19. A composition as in claim 1, wherein the first cross-link moiety does not substantially dissociated upon interaction with the second reagent.

20. An article, comprising:
a composition as in claim 1 at least partially encapsulated by an outer shell.

21-44. (canceled)
